(19) United States
(12) Patent Application Publication
(10) Pub. No.: US 2021/0169959 A1
(43) Pub. Date: Jun. 10, 2021

(54) TERMINALIA FERDINANDIANA EXTRACT AND PRODUCTS CONTAINING EXTRACT OF TERMINALIA FERDINANDIANA FOR ANTIMICROBIAL OR ANTIBACTERIAL APPLICATIONS

(71) Applicant: RISING PHOENIX INDUSTRIES PTY LTD, Mount Lawley (AU)

(72) Inventors: Ian Edwin Cock, Logan Central (AU); David John Boehme, Humpty Doo (AU); Roslyn Anne Miles, Myaree (AU)

(21) Appl. No.: 16/610,867

(22) PCT Filed: May 4, 2018
(86) PCT No.: PCT/AU2018/050408

§ 371 Date: Nov. 4, 2019

(30) Foreign Application Priority Data
May 4, 2017 (AU) 2017901637

(51) Int. Cl. A61K 36/185; A61K 31/366; A61K 31/7048; A61P 33/02; A61P 31/04

(52) U.S. Cl. CPC A61K 36/185; A61K 31/366; A61K 31/7048; A61K 2236/35; A61P 31/04; A61K 2236/333; A61P 33/02

(57) ABSTRACT

A composition or medicament including an extract of Terminalia ferdinadiana (T. ferdinandiana) as an antimicrobial or antibacterial agent for use in treating microbiasl/bacterial infection in humans or animals, preferably B. anthracis or C. perfringens or Giardia infection. The extract may be or include T. ferdinandiana leaf extract. The extract can include at least one antioxidant, such as ellagic acid or trimethyl ellagic acid, and can include at least one tannin and/or at least one flavone and/or chebulic acid, corilagen, chebulinic acid or chebulagic acid and/or at least one flavone or flavinoid.

### FIELD OF THE INVENTION

The present invention relates to natural extracts and/or derivatives of Terminalia ferdinandiana (T. ferdinandiana).

The present invention particularly, though not solely, utilises extracts and/or derivatives of Terminalia ferdinandiana leaf.

The present invention finds application in antibacterial or antimicrobial products or uses, such as for use in treating infections in humans and animals.

### BACKGROUND TO THE INVENTION

Hereinafter, Terminalia ferdinandiana may be referred to as T. ferdinandiana for ease of reference.

T. ferdinandiana is a small, deciduous tree which grows wild extensively throughout the subtropical woodlands of northern tracts of Australia, typically in the Northern Territory and Western Australia.

T. ferdinandiana bears an abundant crop of small plum-like fruits. The fruit is known to have very high vitamin C content, and is a source of antioxidants, folic acid and iron. The fruit and extracts of the fruit are used in foods, dietary supplements and pharmaceuticals.

The commonest use of T. ferdinandiana fruits is for gourmet jams, sauces, juices, ice-cream, cosmetics, flavours and pharmaceuticals.

Examples of cosmetic vehicles for the T. ferdinandiana fruit extract have been proposed in European patent document EP 1581513. Another patent document U.S. Pat. No. 7,175,862 discloses a method of producing a powder containing ascorbic acid (vitamin C), antioxidants and phytochemicals from the fruit of the T. ferdinandiana plant. U.S. Pat. No. 7,175,862 mentions use of the powdered T. ferdinandiana fruit for the reduction of free radicals in the human body.

T. ferdinandiana fruit is also known for having antimicrobial properties. As a native fruit of northern Australia, the fruit has a long history of use by indigenous Australians as a food and a medicinal agent. The fruit was eaten during long hunting trips by indigenous Australians as a source of high nutrition food. The medicinal properties of T. ferdinandiana have not been well understood or fully evaluated.

A study by I. E. Cock and S. Mohanty reporting on an evaluation of the antimicrobial properties of T. ferdinandiana fruit pulp was published in the Pharmacgnosy Journal 2011 [vol 3 I issue 20]. That study focussed on the bacterial growth inhibitory potential of T. ferdinandiana fruit pulp and recognised that further studies were needed to examine other medicinally important bioactivities of T. ferdinandiana fruit.

Despite reported growth inhibitory activity of fruit, numerous pathogens are yet to be evaluated for the ability to inhibit their growth.

In particular, the antibacterial properties of leaf extracts of T. ferdinandiana remain unrealised.

Many bacteria can infect humans and animals. Some bacteria, such as Clostridium perfringens, are anaerobically active. Other bacteria, such as Bacillus anthracis, are aerobically or anaerobically active.

Clostridium perfringens causes myonecrosis, a condition of necrotic damage, specific to muscle tissue. It is often seen in infections with C. perfringens or any of myriad soil-borne anaerobic bacteria. Bacteria cause myonecrosis by specific exotoxins. These microorganisms are opportunistic and, in general, enter the body through significant skin breakage. Gangrenous infection by soil-borne bacteria was common in the combat injuries of soldiers well into the 20th century, because of non-sterile field surgery and the basic nature of care for severe projectile wounds.

Clostridium perfringens (C. perfringens) is an endospore-forming, gram-positive bacterium and the etiological agent of various diseases, including clostridial myonecrosis and enteritis necroticans.

The C. perfringens bacterium grows strictly anaerobically (although it is aero-tolerant) and is found ubiquitously in the environment as part of the natural microbial flora. The bacterium is often also present in the digestive tract of humans and other vertebrates.

Under stresses, such as harsh environmental surroundings or when deprived of necessary nutrients, C. perfringens can produce endospores that place it in a metabolically dormant state as a defence mechanism until conditions are once again favourable for cellular proliferation.

The environmental robustness of the C. perfringens bacterium has significant clinical implications and under anoxic conditions is responsible for a wide variety of diseases, some of which are highly fatal.

Clostridial myonecrosis (or gas gangrene) is a rapidly progressive, highly lethal infection of the skeletal muscle caused by several exotoxin-producing Clostridium species. Though it is caused by a number of species within the Clostridium genus (including C. septicum, C. histolyticum or C. novyi), the predominant cause of gas gangrene is through C. perfringens, which is estimated to be the causative agent in the greatest proportion of documented cases (said to be between 80% and 90% of such cases).

The C. perfringens bacterium is reliant on anaerobic conditions and thus infection occurs primarily in deep tissues, either as a result trauma or post-surgery. Associated exotoxins are subsequently produced and these necrotize the surrounding tissue, resulting in muscular degradation. Unless prompt treatment is administered, later symptoms may include acute renal failure, shock, coma and ultimately death.

Current strategies in the treatment of C. perfringens induced gas gangrene involve a combination of both antibiotic therapy and aggressive surgical debridement.

Without prompt treatment, gas gangrene is highly fatal and thus the removal of necrotized tissues is often necessary to reduce the chance of host death. In recent times there has been an emphasis on producing an effective vaccine, however this is viewed more as a preventative measure than as a curative therapy and thus has no use once infection has initiated. Furthermore, the sporadic, opportunistic nature of the pathogen results in difficulty in predicting who should receive the vaccination.

Thus, the primary method of treatment for gas gangrene currently involves the administration of a combination of penicillin and clindamycin as soon as the infection is detected. Although the bacterium has remained relatively susceptible to antibiotics, reports of antibiotic resistant C. perfringens have emerged and thus there is an ever-increasing need to discover and develop alternative chemotherapeutic options for the treatment of gas gangrene.

Zoonotic infections are diseases that can be transmitted indirectly or directly between humans and animals and are a significant burden from both health and economic standpoints. Such diseases can be spread to humans from both domesticated and wild animals and can be transferred through direct contact, the contamination of drinking water by animal secretions, or the consumption of contaminated meat products. These diseases pose an exceptional set of problems in the control and treatment of infections, as the traditionally effective strategies of herd immunity and isolation of infected individuals are not feasible.

Furthermore, unlike humans who can verbalise otherwise indistinguishable symptoms, infected animals may go unnoticed and further contribute to the spread of disease. From 1940 to 2004, it is thought that approximately 60% of all emerging infectious diseases were of a zoonotic nature with the majority originating in wildlife. Therefore, the development of cross species treatments plays a key role in the effective control and eradication of zoonotic diseases.

By way of example, Bacillus anthracis (B. anthracis), the etiological agent of anthrax, is a sporulating gram-positive bacterium found predominately in soils. Similar to other organisms within the Bacillus genus, B. anthracis is capable of producing endospores that can remain dormant for several years until conditions are again favourable for growth. These spores are metabolically inactive and are capable of surviving environmental conditions that would kill vegetative cells, including temperature, desiccation and enzymatic destruction.

Four forms of human anthrax disease are recognized based on their portal of entry to the human body: 1. Cutaneous, the most common form (95%), causes a localized, inflammatory, black, necrotic lesion (eschar); 2. Inhalation, a rare but highly fatal form, is characterized by flu like symptoms, chest discomfort, diaphoresis, and body aches; 3. Gastrointestinal, a rare but also fatal (causes death to 25%) type, results from ingestion of anthrax spores. Symptoms include: fever and chills, swelling of neck, painful swallowing, hoarseness, nausea and vomiting (especially bloody vomiting), diarrhea, flushing and red eyes, and swelling of abdomen; 4. Injection, symptoms are similar to those of cutaneous anthrax, but injection anthrax can spread throughout the body faster and can be harder to recognize and treat compared to cutaneous anthrax.

Although the vegetative B. anthracis cells produce the toxins associated with the disease, infection is generally initiated when spores are introduced into a host through inhalation, ingestion or via direct contact with open wounds. Once internalised, the spores revert to viable cells, proliferate and begin producing the deadly anthrax toxins.

The disease has been controlled to varying degrees internationally through careful monitoring and strong eradication measures. However, anthrax is endemic worldwide and is often fatal if infection occurs.

Current strategies in the treatment of anthrax typically involve a combination of antibiotic therapies to fight infection, as well as supportive care to manage associated symptoms.

The administration of intravenous or oral antibiotics are generally effective in the management of anthrax, however there is always an inherent risk that the bacteria may develop drug resistance. As such, the discovery of new drugs is of significant importance, either through the design and synthesis of new compounds, or through the investigation of antimicrobials within pre-existing natural assets.

Giardiasis is a major cause of infectious diarrhoea in humans and livestock worldwide. Giardiasis is caused by gastrointestinal infections of protozoal parasites of the genus Giardia. There is a limited range of drugs available for chemotherapeutic treatment of this disease, and they are only used after clinical diagnosis and not for prophylaxis.

The majority of these drugs are ineffective against some life stages of the Giardia protozoa, are toxic, have unpleasant side effects and may have limited availability in developing countries. Treatment failure and parasite resistance highlight the importance to develop new chemotherapeutic treatments for giardiasis with greater efficacy and less severe side effects.

Treatment of giardiasis using natural plant derived compounds is an attractive prospect as the medicinal qualities of plants can be very efficacious.

The antimicrobial effects of medicinal plants have long been recognised by many cultures and phytochemical analysis to identify the active compounds offers promise in the development of new antimicrobial agents, such as for treatment of giardiasis and anti-B. anthracis agents.

Thus, the development of natural assets provides great potential in the discovery of compounds effective in managing disease causing microbes, such as bacteria causing anthrax, giardiasis or clostridium.

It is with such aforementioned bacteria and bacterial infections in mind that the present invention has been developed.

### SUMMARY OF THE INVENTION

According to one or more forms of the present invention and methods/tests assessing T. ferdinandiana fruit and leaf extracts, it has been realised that products containing T. ferdinandiana leaf extract are efficacious in inhibiting the growth of microbes, such as bacteria, e.g. the gram-positive anaerobic bacterium Clostridium perfringens (C. perfringens) or Bacillus anthracis (B. anthracis) or the genus Giardia, such as Giardia duodenalis.

An aspect of the present invention provides an extract of Terminalia ferdinandiana (T. ferdinandiana) for use in a medicament for treatment of microbial or bacterial infection in humans or animals.

A further aspect of the present invention provides a medicament including extract of Terminalia ferdinadiana (T. ferdinandiana).

Preferably, the extract includes extract of T. ferdinandiana leaf.

A composition for use in a medicament for use in treating microbial or bacterial infection in humans or animals, the medicament containing an extract derived from Terminalia ferdinandiana (T. ferdinandiana) leaf as an antibacterial agent.

The medicament or composition may be provided for use as one or more pills, tablets, capsules or in liquid form.

The medicament may include an extract of T. ferdinandiana fruit in addition to the extract of T. ferdinandiana leaf.

Preferably the T. ferdinandiana leaf extract includes one or more of a methanolic/ethanolic extract, aqueous extract, ethyl acetate extract, chloroform extract or hexane extract.

The T. ferdinandiana leaf extract may include a proportion of at least one antioxidant.

The at least one antioxidant may include one or more of an ellagic acid or trimethyl ellagic acid.

The extract, composition or medicament provided as an antimicrobial agent for use n treating bacterial infection in humans or animals.

Preferably the extract, medicament or composition is provided in pill form, capsule form, or as a liquid, including the extract of T. ferdinandiana leaf.

The extract, medicament or composition may include at least one tannin and/or at least one flavone.

The extract, medicament or composition may include one of or a combination of two or more of, chebulic acid, corilagen, chebulinic acid and chebulagic acid.

The extract, medicament or composition may include at least one flavone or flavinoid.

The extract, medicament or composition may include one or more antioxidants. The at least one antioxidant may include an ellagic acid. The ellagic may include ellagic acid dehydrate and/or trimethyl ellagic acid.

A further aspect of the present invention provides an antimicrobial composition containing an extract derived from Terminalia ferdinandiana (T. ferdinandiana) leaf.

Preferably, the extract or medicament is provided in a medicament for use in treating B. anthracis or C. perfringens or Giardia infection in humans or animals.

A further aspect of the present invention provides for use of an extract of Terminalia ferdinandiana (T. ferdinandiana) in the preparation of a medicament or composition for use in treating microbial or bacterial infection in humans or animals. The use may include the extract including T. ferdinandiana leaf extract.

### BRIEF DESCRIPTION OF THE DRAWINGS

One or more embodiments of the present invention will hereinafter be described with reference to the accompanying Figures and Tables, in which:

FIG. 1 shows a chart of growth inhibitory activity of T. ferdinandiana fruit and leaf plant extracts against the C. perfringens environmental isolate measured as zones of inhibition (mm).

FIG. 2 shows a chart of the lethality of the Australian plant extracts (2000 μg/mL) and the potassium dichromate control (1000 μg/mL) towards Artemia franciscana nauplii after 24 h exposure.

FIG. 3a shows positive and FIG. 3b negative ion RP-HPLC total compound chromatograms (TCC) of 2 μl injections of T. ferdinandiana leaf methanolic extract.

FIG. 4a shows positive and FIG. 4b negative ion RP-HPLC total compound chromatograms (TCC) of 2 μl injections of T. ferdinandiana leaf ethyl acetate extract.

FIG. 5 shows chemical structures of T. ferdinandiana leaf tannin compounds detected in the methanolic and/or ethyl acetate extracts: (a) chebulic acid; (b) protocatechuic acid; (c) ellagic acid dihydrate; (d) punicalagin; (e) ellagic acid; (f) chebulagic acid; (g) castalagin; (h) corilagin; (i) punicalin; (j) chebulinic acid; (k) punicalin; (l-m) trimethylellagic acid isomers.

FIG. 6 shows a chart of growth inhibitory activity of T. ferdinandiana plant extracts against the B. anthracis environmental isolate measured as zones of inhibition (mm). FW=aqueous T. ferdinandiana fruit extract; FM=methanolic T. ferdinandiana fruit extract; FC=chloroform T. ferdinandiana fruit extract; FH=hexane T. ferdinandiana fruit extract; FE=ethyl acetate T. ferdinandiana fruit extract; LW=aqueous T. ferdinandiana leaf extract; LM=methanolic T. ferdinandiana leaf extract; LC=chloroform T. ferdinandiana leaf extract; LH=hexane T. ferdinandiana leaf extract; LE=ethyl acetate T. ferdinandiana leaf extract; PC=penicillin (2 μg); AMP=ampicillin (10 μg). Results are expressed as mean zones of inhibition±SEM.

FIG. 7 shows a chart of the lethality of the T. ferdinandiana fruit and leaf plant extracts (2000 pg/mL) and the potassium dichromate control (1000 μg/mL) towards Artemia franciscana nauplii after 24 hour exposure. FW=aqueous T. ferdinandiana fruit extract; FM=methanolic T. ferdinandiana fruit extract; FC=chloroform T. ferdinandiana fruit extract; FH=hexane T. ferdinandiana fruit extract; FE=ethyl acetate T. ferdinandiana fruit extract; LW=aqueous T. ferdinandiana leaf extract; LM=methanolic T. ferdinandiana leaf extract; LC=chloroform T. ferdinandiana leaf extract; LH=hexane T. ferdinandiana leaf extract; LE=ethyl acetate T. ferdinandiana leaf extract; PC=potassium dichromate control; SW=seawater control. Results are expressed as mean % mortality±SEM.

FIG. 8 shows a chart representing a head space gas chromatogram of 0.5 μL injections of T. ferdinandiana ethyl acetate fruit extract. The extract were dried and resuspended in methanol for analysis.

FIG. 9 shows a chart representing a head space gas chromatogram of 0.5 μL injections of methanolic T. ferdinandiana leaf extract. The extract were dried and resuspended in methanol for analysis.

FIGS. 10a to 10n show examples of compounds present in the leaf and fruit extracts, such as one or more furans and/or tannins.

FIGS. 11a to 11k show examples of compounds present in T. ferdinandiana leaf with properties consistent with anti-giardial activity according to at least one embodiment of the present invention.

FIG. 12 shows inhibitory activity of the T. ferdinandiana extracts and pure compounds against three strains of Giardia duodenalis trophozoites measured as a percentage the untreated control.

FIGS. 13a to 13c show Isobolograms for combinations of gallic acid and ascorbic acid tested at various ratios against (a) the sheep S2, (b) reference metronidazole sensitive (ATCC203333) and (c) reference metronidazole resistant (ATCC PRA-251) G. duodenalis strains.

FIGS. 14a to 14c show isobologramss of the association between the growth inhibitory activity and the DPGA axis.

### DESCRIPTION OF PREFERRED EMBODIMENT

One or more methods for obtaining extract(s) and/or derivatives of T. ferdinandiana for one or more embodiments of the present invention will hereinafter be described. However, it is to be understood and appreciated that the generality of the present invention is not to be limited by the specific scope of the following specific description.

Solvent extracts and aqueous extracts were prepared using the fruit and the leaf of T. ferdinandiana.

Clostridium Perfringens (C. Perfringens)

T. ferdinandiana fruit and leaf solvent and aqueous extracts were investigated for growth inhibitory activity by disc diffusion assay against a clinical strain of C. perfringens.

Their minimum inhibitory concentration (MIC) values were determined to quantify and compare their efficacies.

Toxicity was determined using the Artemia franciscana nauplii bioassay. Active extracts were analysed by non-targeted High Performance Liquid Chromatography-Quadrupole Time-of-Flight (HPLC-QTOF) mass spectroscopy (with screening against 3 compound databases) for the identification and characterisation of individual components in the crude T. ferdinandiana fruit and leaf extracts.

Methanolic and aqueous T. ferdinandiana fruit and leaf extracts, as well as the leaf ethyl acetate extract, displayed growth inhibitory activity in the disc diffusion assay against C. perfringens.

The leaf extracts were generally more potent growth inhibitors than the corresponding fruit extracts, although the aqueous fruit extract had substantially greater efficacy than the aqueous leaf extract.

The methanolic and ethyl acetate leaf extracts were particularly potent growth inhibitors, with MIC values of 206 and 117 μg/ml respectively.

The fruit methanolic extract also displayed good efficacy, with an MIC of 716 μg/ml. I

In contrast, the chloroform and hexane extracts of both fruit and leaf were completely devoid of growth inhibitory activity.

All T. ferdinandiana extracts were either nontoxic or of low toxicity in the Artemia fransiscana bioassay. Non-biased phytochemical analysis of the methanolic and ethyl acetate leaf extracts revealed the presence of high relative levels of a diversity of gallo- and ellagi-tannins.

The low toxicity of the T. ferdinandiana extracts and the potent growth inhibitory bioactivity of the leaf methanolic and ethyl acetate extracts against C. perfringens indicates their potential as medicinal agents in the treatment and prevention of clostridial myonecrosis and enteritis necroticans. Metabolomic profiling studies indicate that these extracts contained a diversity of tannins.

Plant source and extraction: T. ferdinandiana fruit, leaves and pulp were obtained. The pulp was frozen prior to transport and kept at −10° C. until processed. The leaves were extensively dehydrated in a dehydrator and the desiccated material was stored at −30° C. The fruit and leaf materials were thoroughly dried and ground into a coarse powder prior to use. A mass of 1g of ground powder was extensively extracted in 50 mL of either de-ionised water, methanol, chloroform, hexane or ethyl acetate for 24 h at 4° C. with gentle agitation. The extracts were filtered through filter paper (Whatman No. 54) and air dried at room temperature. The aqueous extract was lyophilised by rotary evaporation in a concentrator. The resultant pellets were dissolved in 10 mL deionised water (containing 0.5% DMSO). The extract was passed through a 0.22 μm filter (Sarstedt) and stored at 4° C. until used.

Qualitative phytochemical studies: Phytochemical analysis of the extracts for the presence of triterpenoids, tannins, saponins, phytosteroids, phenolic compounds, flavonoids, cardiac glycosides, anthraquinones and alkaloids were conducted by previously described assays.

Antioxidant capacity: The antioxidant capacity of each sample was assessed using a modified 2,2-diphenyl-1-picrylhydrazyl (DPPH) free radical scavenging method. Ascorbic acid (0-25 μg per well) was used as a reference and the absorbance was measured and recorded at 515 nm. All tests were completed alongside controls on each plate and all were performed in triplicate. The antioxidant capacity based on DPPH free radical scavenging ability was determined for each extract and expressed as pg ascorbic acid equivalents per gram of original plant material extracted.

Antibacterial screening: Clinical Clostridium perfringens screening: A clinical strain of C. perfringens was obtained.

Evaluation of antimicrobial activity: Antimicrobial activity of all of the leaf and fruit T. ferdinandiana plant extracts was determined using a modified disc diffusion assay. Briefly, 100 μL of C. perfringens was grown in 10 mL of fresh thioglycollate media until they reached a count of ˜10⁸ cells/mL. A volume of 100 μL of the bacterial suspension was spread onto nutrient agar plates and extracts were tested for antibacterial activity using 6 mm sterilised filter paper discs. Discs were impregnated with 10 μL of T. ferdinandiana extracts, allowed to dry and placed onto the inoculated plates. The plates were allowed to stand at 4° C. for 2 hours before incubation at 30° C. for 24 hours. The diameters of the inhibition zones were measured to the closest whole millimetre. Each assay was performed in at least triplicate. Mean values (±SEM) are reported herein. Standard discs of penicillin (2 μg) and ampicillin (10 μg) were obtained and used as positive controls to compare antibacterial activity. Filter discs impregnated with 10 μL of distilled water were used as a negative control.

Minimum inhibitory concentration (MIC) determination: The minimum inhibitory concentrations (MIC) of the extracts was determined as previously described. Briefly, the T. ferdinandiana fruit and leaf plant extracts were diluted in deionised water and tested across a range of concentrations. Discs were impregnated with 10 μL of the extract dilutions, allowed to dry and placed onto inoculated plates. The assay was performed as outlined above and graphs of the zone of inhibition versus concentration were plotted. Linear regression was used to determine MIC values.

Toxicity screening: Reference toxin for toxicity screening: Potassium dichromate (K₂Cr₂O₇) was prepared in distilled water (4 mg/mL) and serially diluted in artificial seawater for use in the Artemia franciscana nauplii bioassay.

Artemia Franciscana Nauplii Toxicity Screening

Toxicity was tested using a modified Artermia franciscana nauplii lethality assay. Briefly, 400 μL of seawater containing ˜43 (mean 43.2, n=155, SD 14.5) A. franciscana nauplii were added to wells of a 48 well plate and immediately used in the bioassay. Volumes of 400 μL of reference toxin or the diluted plant extracts were transferred to the wells and incubated at 25±1° C. under artificial light (1000 Lux). A negative control (400 μL seawater) was run in triplicate for each plate. All treatments were performed in at least triplicate. The wells were checked at regular intervals and the number of dead counted. The nauplii were deemed dead if no movement of the appendages was detected within 10 seconds. After 24 h, all nauplii were sacrificed and counted to determine the total % mortality per well. The LC50 with 95% confidence limits for each treatment was calculated using probit analysis.

Non-targeted HPLC-MS QTOF analysis: For chromatographic separations, 2 μL of sample was injected onto an HPLC system fitted with a column (2.1×100 mm, 1.8 μm particle size). The mobile phases consisted of (A) ultrapure water and (B) 95:5 acetonitrile/water at a flow rate of 0.7 mL/min. Both mobile phases were modified with 0.1% (v/v) glacial acetic acid for mass spectrometry analysis in positive mode and with 5 mM ammonium acetate for analysis in negative mode. The chromatographic conditions utilised for the study consisted of the first 5 min run isocratically at 5% B, a gradient of (B) from 5% to 100% was applied from 5 min to 30 min, followed by 3 min isocratically at 100%. Mass spectrometry analysis was performed on a quadrapole time-of-flight mass spectrometer (QTOF MS) fitted with an electrospray ionisation source in both positive and negative mode.

Data was analysed using known qualitative analysis software. Blanks using each of the solvent extraction systems were analysed using the ‘Find by Molecular Feature’ algorithm in the software package to generate a compound list of molecules with abundances greater than 10,000 counts. This was then used as an exclusion list to eliminate background contaminant compounds from the analysis of the extracts. Each extract was then analysed using the same parameters using the ‘Find by Molecular Feature’ function to generate a putative list of compounds in the extracts. Compound lists were then screened against three accurate mass databases; a database of known plant compounds of therapeutic importance generated specifically for this study (800 compounds); a known metabolomics database (24,768 compounds); and a known forensic toxicology database (7,509 compounds). Empirical formula for unidentified compounds was determined using the Find Formula function in the software package.

Statistical analysis: Data is expressed as the mean±SEM of at least three independent experiments.

Liquid extraction yields and qualitative phytochemical screening

T. ferdinandiana plant extractions (1 g) with various solvents yielded dried plant extracts ranging from 18 mg to 483 mg (fruit extracts) and 58 mg to 471 mg (leaf extracts) (see Table 1).

TABLE 1
Table 1: The mass of dried extracted material, the concentration after re-suspension in deionised water,
qualitative phytochemical screenings and antioxidant capacities of the T. ferdinandiana extracts:

| | | Concentration | Antioxidant Capacity | | | | | |
|---|---|---|---|---|---|---|---|---|
| | Mass of Dried | of Resuspended | (mg Ascorbic Acid | Total | Water Soluble | Water Insoluble | Cardiac | |
| Extract | Extract (mg) | Extract (mg/mL) | Equivalency) | Phenolics | Phenolics | Phenolics | Glycosides | Saponins |
| KFW | 483 | 48.3 | 264 | +++ | +++ | +++ | − | + |
| KFM | 359 | 35.9 | 660 | +++ | +++ | +++ | − | ++ |
| KFC | 62 | 6.2 | 7 | + | − | − | − | − |
| KFH | 18 | 1.8 | 1 | − | − | − | − | − |
| KFE | 30 | 3 | 39 | ++ | ++ | + | − | + |
| KLW | 471 | 47.1 | 340 | +++ | +++ | +++ | ++ | +++ |
| KLM | 331 | 33.1 | 150 | +++ | +++ | +++ | +++ | ++ |
| KLC | 59 | 5.9 | 5 | + | − | − | − | − |
| KLH | 58 | 5.8 | 0.4 | + | − | − | − | − |
| KLE | 59 | 5.9 | 22 | +++ | +++ | +++ | − | − |

| | | | Alkaloids | Alkaloids | | | Free | Combined |
|---|---|---|---|---|---|---|---|---|
| Extract | Triterpenes | Phytosteroids | (Mayer Test) | (Wagner Test) | Flavonoids | Tannins | Anthraquinones | Anthraquinones |
| KFW | − | − | − | − | +++ | ++ | − | − |
| KFM | + | − | + | + | +++ | ++ | − | − |
| KFC | − | − | − | − | − | − | − | − |
| KFH | − | − | − | − | − | − | − | − |
| KFE | ++ | − | − | − | ++ | − | − | − |
| KLW | ++ | − | − | − | ++ | +++ | + | + |
| KLM | + | − | + | + | ++ | +++ | + | + |
| KLC | − | − | − | − | − | − | − | − |
| KLH | − | − | − | − | ++ | + | − | − |
| KLE | − | − | − | − | ++ | ++ | − | − |

In Table 1 above, + + + indicates a large response; + + indicates a moderate response; + indicates a minor response; − indicates no response in the assay. KFW=aqueous T. ferdinandiana fruit extract; KFM=methanolic T. ferdinandiana fruit extract; KFC=chloroform T. ferdinandiana fruit extract; KFH=hexane T. ferdinandiana fruit extract; KFE=ethyl acetate T. ferdinandiana fruit extract; KLW=aqueous T. ferdinandiana leaf extract; KLM=methanolic T. ferdinandiana leaf extract; KLC=chloroform T. ferdinandiana leaf extract; KLH=hexane T. ferdinandiana leaf extract; KLE=ethyl acetate T. ferdinandiana leaf extract. Antioxidant capacity was determined by DPPH reduction and is expressed as mg ascorbic acid equivalence per g plant material extracted.

Aqueous and methanolic extracts provided significantly greater yields of extracted material relative to the chloroform, ethyl acetate and hexane counterparts, which gave low to moderate yields. The dried extracts were resuspended in 10 mL of deionised water (containing 1% DMSO), resulting in the concentrations presented in Table 1.

Antioxidant content: Antioxidant capacity for the plant extracts (Table 1) ranged from 0.4 mg (hexane leaf extract) to a high of 660 mg ascorbic acid equivalence per gram of dried plant material extracted (methanolic fruit extract). The aqueous and methanolic extracts generally had higher antioxidant capacities than the corresponding chloroform, hexane and ethyl acetate extracts.

Antimicrobial activity: To determine the ability of the fruit and leaf crude extracts to inhibit C. perfringens growth, 10 μL of each extract was screened using a disc diffusion assay.

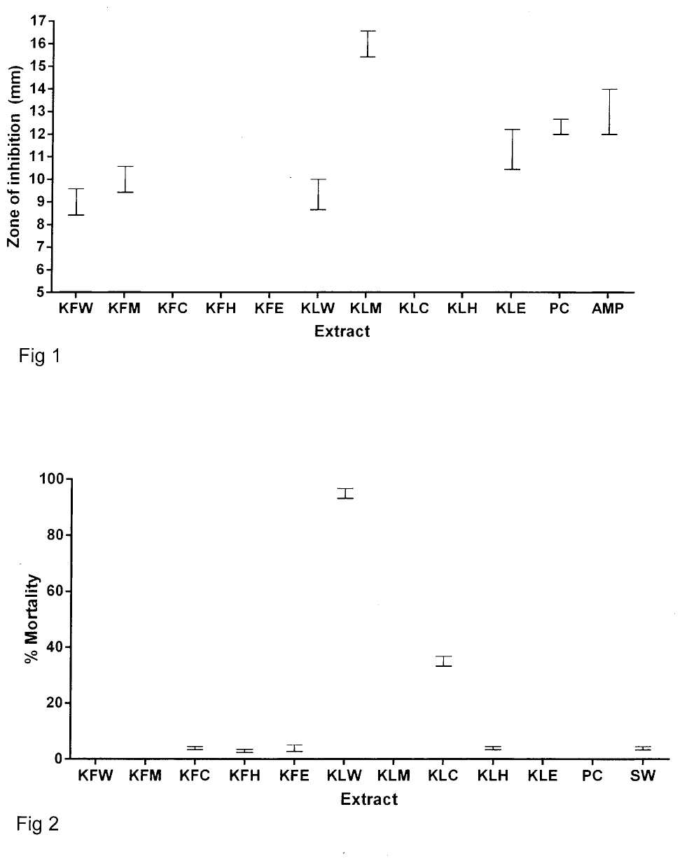

As shown in the chart in FIG. 1, Bacterial growth was strongly inhibited by 5 of the 10 extracts screened (50%).

The methanolic leaf extract was the most potent inhibitor of growth (as judged by zone of inhibition), with inhibition zones of 16±0.6 mm. This compares favourably with the penicillin (2 μg) and ampicillin controls (10 μg), with the zones of inhibition of 12.3±0.3 and 13±1.0 mm respectively.

The methanolic fruit extract as well as both the aqueous and ethyl acetate leaf extracts also displayed good inhibition of C. perfringens growth, with ≥9 mm zones of inhibition.

Typically, the leaf extracts were more potent inhibitors of C. perfringens growth than were their corresponding fruit extract counterparts.

FIG. 1 shows a chart of growth inhibitory activity of T. ferdinandiana fruit and leaf plant extracts against the C. perfringens environmental isolate measured as zones of inhibition (mm). KFW=aqueous T. ferdinandiana fruit extract; KFM=methanolic T. ferdinandiana fruit extract; KFC=chloroform T. ferdinandiana fruit extract; KFH=hexane T. ferdinandiana fruit extract; KFE=ethyl acetate T. ferdinandiana fruit extract; KLW=aqueous T. ferdinandiana leaf extract; KLM=methanolic T. ferdinandiana leaf extract; KLC=chloroform T. ferdinandiana leaf extract; KLH=hexane T. ferdinandiana leaf extract; KLE=ethyl acetate T. ferdinandiana leaf extract; PC=penicillin (2 μg); AMP =ampicillin (10 μg). Results are expressed as mean zones of inhibition±SEM.

The antimicrobial efficacy was further quantified through the determination of MIC values against the T. ferdinandiana extracts (Table 2).

Table 2 below shows minimum inhibitory concentration (μg/mL) of the T. ferdinandiana fruit and leaf extracts and LC50 values (μg/mL) in the Artemia nauplii bioassay (Numbers indicate the mean MIC and LC50 values of triplicate determinations.—indicates no inhibition):

TABLE 2

| Extract | MIC | LC₅₀ |
|---|---|---|
| aqueous fruit extract | 1192 | 2,080 |
| methanolic fruit extract | 716 | 2,115 |
| chloroform fruit extract | — | — |
| hexane fruit extract | — | — |
| ethyl acetate fruit extract | — | — |
| aqueous leaf extract | 3125 | 1,330 |
| methanolic leaf extract | 206 | 1,133 |
| chloroform leaf extract | — | — |
| hexane leaf extract | — | — |
| ethyl acetate leaf extract | 117 | 767 |

The aqueous and methanolic extracts (both fruit and leaf), as well as the leaf ethyl acetate extract, were effective at inhibiting C. perfringens growth, with MIC values generally <1000 μg/ml (<10 μg impregnated in the disc).

The methanolic and ethyl acetate leaf extracts were particularly potent, with MIC values of 206 μg/mL (approximately 2.1 μg infused into the disc) and 117 μg/mL (approximately 1.2 μg infused into the disc) respectively.

These results compare well with the growth inhibitory activity of the penicillin and ampicillin controls which were tested at 2 μg and 10 μg respectively.

The methanolic fruit extract was also a potent C. perfringens growth inhibitor (MIC value of 716 μg/ml).

Whilst less potent, the aqueous fruit extract also displayed good growth inhibitory activity (MIC values of 1192 μg/ml).

In contrast, both chloroform and hexane extracts, as well as the fruit ethyl acetate extract, were not active, or were of only low efficacy in the assay.

Quantification of toxicity: All extracts were initially screened in the assay at 2000 μg/mL (see FIG. 2).

As a reference toxin, potassium dichromate was also tested in the bioassay. The potassium dichromate reference toxin was rapid in its onset of mortality, inducing nauplii death within the first 3 h of exposure and 100% mortality evident within 4-5 h (results omitted).

All aqueous and methanolic extracts as well as the ethyl acetate leaf extract showed >90% mortality rates at 24 h.

The other extracts showed <10% mortality rates at 24 h, with the exception of the chloroform leaf extract.

To further quantify the effects of toxin concentration on the initiation of mortality, the extracts were serially diluted in artificial seawater to test across a series of concentrations in the Artemia franciscana nauplii bioassay at 24 hours. The LC₅₀ values of the T. ferdinandiana extracts towards A. franciscana are presented in Table 2. No LC₅₀ values are reported in either of the hexane or chloroform extracts, nor for the ethyl acetate fruit extract, as <50% mortality was seen in all tested concentrations.

Extracts with an LC₅₀ greater than 1000 μg/ml towards Artemia nauplii have been defined as being nontoxic in this assay. As only the ethyl acetate fruit extract had an LC₅₀ value of <1000 μg/ml, all other extracts were considered nontoxic. Whilst the LC₅₀ value for the ethyl acetate leaf extract is <1000 μg/ml, a value of 767 μg/ml indicates low to moderate toxicity.

FIG. 2 shows a chart of the lethality of the Australian plant extracts (2000 μg/mL) and the potassium dichromate control (1000 μg/mL) towards Artemia franciscana nauplii after 24 h exposure. KFW=aqueous T. ferdinandiana fruit extract; KFM=methanolic T. ferdinandiana fruit extract; KFC=chloroform T. ferdinandiana fruit extract; KFH=hexane T. ferdinandiana fruit extract; KFE=ethyl acetate T. ferdinandiana fruit extract; KLW=aqueous T. ferdinandiana leaf extract; KLM=methanolic T. ferdinandiana leaf extract; KLC=chloroform T. ferdinandiana leaf extract; KLH=hexane T. ferdinandiana leaf extract; KLE=ethyl acetate T. ferdinandiana leaf extract; PC=potassium dichromate control; SW=seawater control. Results are expressed as mean % mortality±SEM.

It will be appreciated that methanolic extracts include ethanolic extracts.

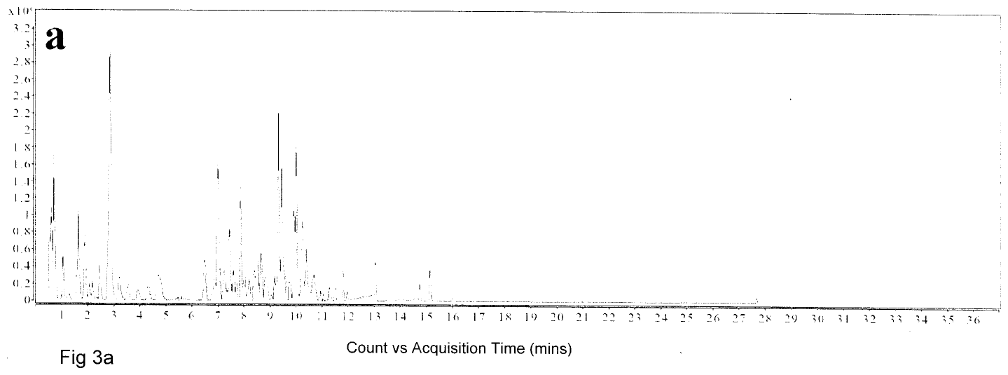

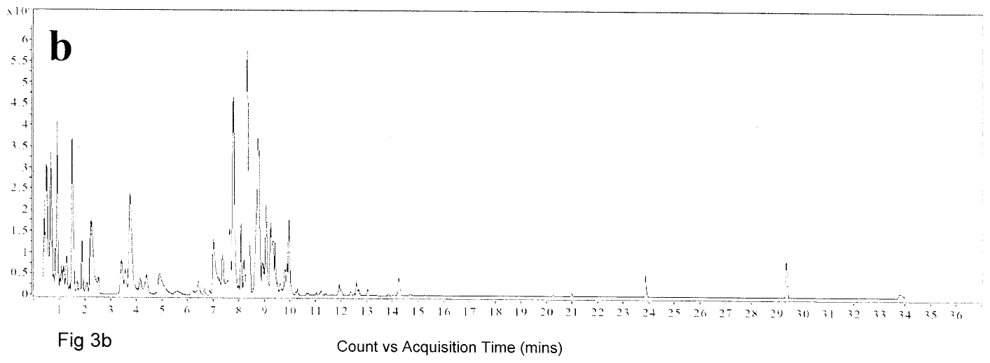

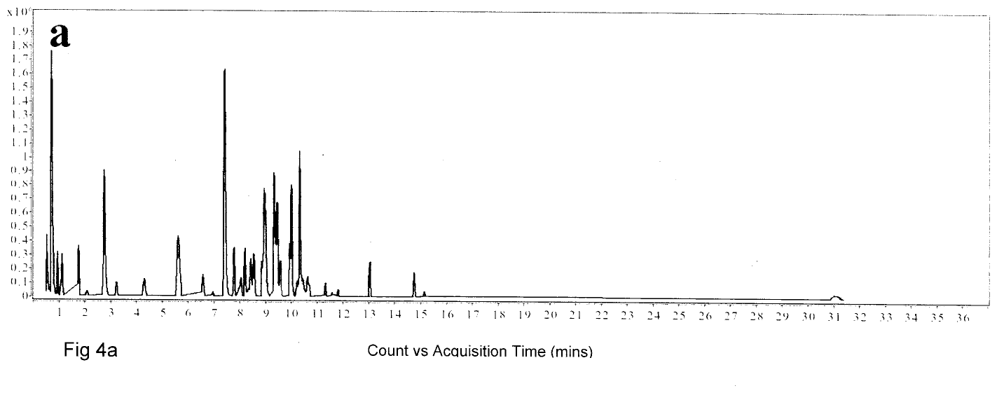

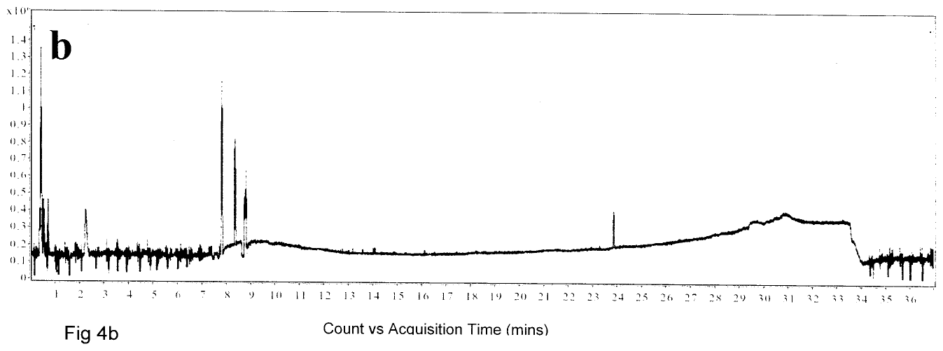

HPLC-MS QTOF analysis: As the methanolic and ethyl acetate leaf extracts had the greatest antibacterial efficacy (as determined by MIC), they were deemed the most promising extracts for further phytochemical analysis. Optimised HPLC-MS QTOF parameters used previously for the analysis of T. ferdinandiana leaf extracts were also used for the determination of the methanolic and ethyl acetate leaf extract compound profiles. The total compound chromatograms of the methanolic and ethyl acetate extracts are presented in FIGS. 3a, 3b and 4a, 4b respectively.

The T. ferdinandiana methanolic extract positive (FIG. 3a) and negative ion (FIG. 3b) total compound chromatogram chromatograms revealed multiple overlapping peaks in the early stages of the chromatogram corresponding to the elution of polar compounds.

Most of the extract compounds had eluted within 12 minutes of the chromatogram (corresponding to approximately 32% acetonitrile).

However, several prominent peaks between 12 and 16 min in both chromatograms, and between 24 and 30 minutes (51-66% acetonitrile) indicates the broad spread of polarities of the compounds in this extract.

The leaf ethyl acetate positive ion (FIG. 4a) chromatogram had a similar elution profile to the corresponding methanolic extract, albeit with fewer peaks evident.

Many of the peaks in this chromatogram corresponded to peaks at similar elution volumes in the methanolic extract, indicating that many compounds were extracted by both solvents.

In contrast, much fewer peaks were evident in the leaf ethyl acetate negative ion chromatogram (FIG. 4b).

However, this chromatogram had significant background absorbance levels than the positive ion chromatogram due to ionisation of negative ions in this mode, possibly masking the signals for some peaks.

FIG. 4a shows positive and FIG. 4b negative ion RP-HPLC total compound chromatograms (TCC) of 2 μl injections of T. ferdinandiana leaf ethyl acetate extract.

In total, fifty-four unique mass signals were noted for the T. ferdinandiana leaf methanolic and/or ethyl acetate extracts (Table 3).

All of the fifty-four unique molecular mass signals detected were putatively identified by comparison to the ‘Metlin’ metabolomics, forensic toxicology (Agilent) and phytochemicals (developed in this laboratory) databases.

Seventeen and eight compounds were detected only in the methanolic and ethyl acetate extracts respectively. The remaining twenty-nine compounds were present in both extracts.

The diversity of tannin compounds is noteworthy, with fourteen tannin compounds putatively identified across the methanolic and ethyl acetate leaf extracts.

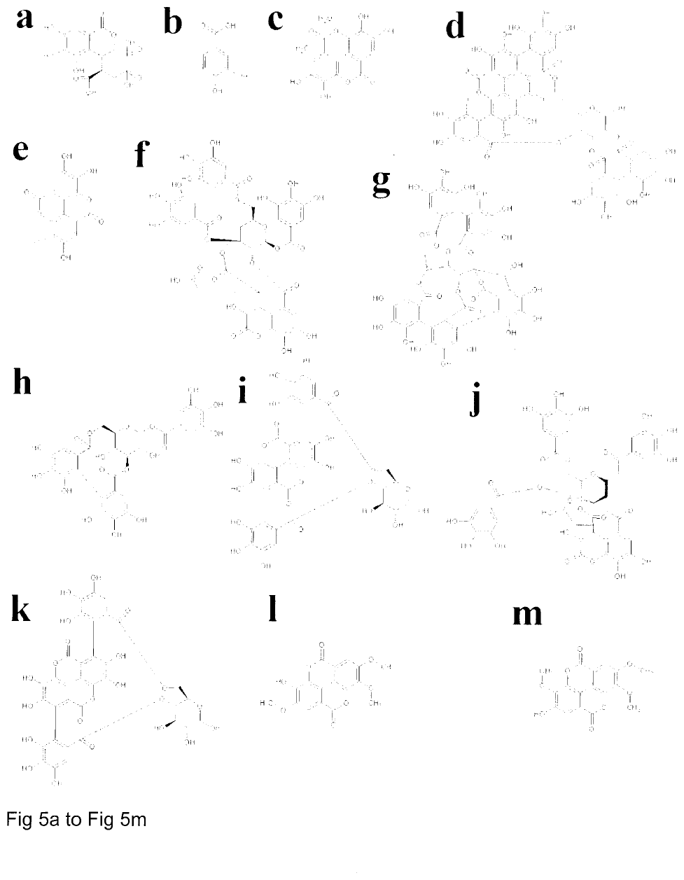

In particular, chebulic acid (FIG. 5a), protocatechuic acid (FIG. 5b), ellagic acid dehydrate (FIG. 5c), punicalagin (FIG. 5d), ellagic acid (FIG. 5e), chebulagic acid (FIG. 5f), castalagin (FIG. 5g), corilagin (FIG. 5h), punicalin (FIG. 5i), chebulinic acid (FIG. 5j), punicalin (FIG. 5k), trimethylellagic acid isomers (FIG. 5l and FIG. 5m) were putatively identified.

Table 3 shows qualitative HPLC-MS/MS analysis of the T. ferdinandiana leaf methanolic and ethyl acetate extracts, elucidation of empirical formulas and putative identification of the compound.

TABLE 3

| Name | Formula | Mass | RT | KLM | KLE |
|---|---|---|---|---|---|
| Chebulic acid (isomer 1) | C₁₄ H₁₂ O₁₁ | 356.0395 | 0.363 | − | +/− |
| Shikimic acid | C₇ H₁₀ O₅ | 174.0542 | 0.403 | − | − |
| Theophylline | C₇ H₈ N₄ O₂ | 180.0649 | 0.424 | − | − |
| (1S,5R)-4-Oxo-6,8- | C₇ H₆ O₅ | 170.0221 | 0.484 | − | |
| dioxabicyclo[3.2.1]oct- | | | | | |
| 2-ene-2-carboxylic acid | | | | | |
| Mannitol | C₆ H₁₄ O₆ | 182.0793 | 0.505 | | + |
| Diprophylline | C₁₀ H₁₄ N₄ O₄ | 254.1012 | 0.512 | | + |
| Protocatechuic acid | C₇ H₆ O₄ | 154.0272 | 0.522 | | − |
| Propionylglycine methyl ester | C₆ H₁₁ N O₃ | 145.0744 | 0.529 | + | |
| Naphtho [2″,3″: 4′,5′] | C₁₉ H₁₀ N₄ S | 326.0645 | 0.621 | − | |
| imidazo [2′,1′: 2,3] | | | | | |
| [1,3] thiazolo [4,5-b] quinoxaline | | | | | |
| Vanilpyruvic acid | C₁₀ H₁₀ O₅ | 210.0529 | 0.632 | | − |
| Valdipromide | C₁₁ H₂₃N O | 185.1785 | 0.91 | | + |
| Ellagic acid dihydrate | C₁₄ H₁₀ O₁₀ | 338.0285 | 1.067 | +/− | + |
| Punicalagin | C₄₈ H₂₈ O₃₀ | 1084.065 | 1.157 | − | |
| Chebulic acid (isomer 2) | C₁₄ H₁₂ O₁₁ | 356.0388 | 1.533 | +/− | + |
| Phloroglucinol | C₆ H₆ O₃ | 126.0322 | 1.605 | + | |
| Phosphoribosylaminoimidazole | C₁₃ H₁₉ N₄ O₁₂ P | 454.0751 | 2.489 | − | |
| succinocarboxamide (SAICAR) | | | | | |
| 2-Cyclohexylpiperidine oxalate | C₁₃ H₂₃ N O₄ | 257.163 | 3.268 | | + |
| (2-Methyl-4-oxo-4H-pyran-3-yloxy)- | C₈ H₈ O₅ | 184.0377 | 3.745 | − | |
| acetic acid | | | | | |
| Ellagic acid | C₁₄ H₆ O₈ | 302.0073 | 4.372 | +/− | + |
| (2-Methyl-4-oxo-4H-pyran-3-yloxy)- | C₈ H₈ O₅ | 184.0374 | 4.788 | | |
| acetic acid | | | | | |
| 1α,25-Dihydroxy-26,27-dimethyl- | C₂₉ H₄₄ O₃ | 440.3262 | 6.929 | | + |
| 22,22,23,23-tetradehydrovitamin D3 | | | | | |
| Chebulagic acid (isomer 1) | C₄₁ H₃₀ O₂₇ | 954.0979 | 7.629 | − | − |
| Castalagin (isomer 1) | C₄₁ H₂₆ O₂₆ | 934.0719 | 7.671 | − | − |
| Corilagin | C₂₇ H₂₂ O₁₈ | 634.0815 | 7.773 | +/− | +/− |
| 8-Epiiridotrial glucoside | C₁₆ H₂₄ O₈ | 344.1475 | 8.42 | − | |
| Punicalin | C₃₄ H₂₂ O₂₂ | 782.0619 | 8.498 | + | + |
| Chebulinic acid (isomer 1) | C₄₁ H₃₂ O₂₇ | 956.1131 | 8.602 | − | − |
| Luteolin (isomer 1) | C₂₁ H₂₀ O₁₁ | 448.1018 | 8.726 | +/− | +/− |
| Castalagin (isomer 2) | C₄₁ H₂₆ O₂₆ | 934.0705 | 8.767 | − | − |
| Vitexin | C₂₁ H₂₀ O₁₀ | 432.1067 | 9.201 | +/− | +/− |
| Exifone | C₁₃ H₁₀ O₇ | 278.0431 | 9.314 | +/− | +/− |
| Rutin | C₂₇ H₃₀ O₁₆ | 610.1542 | 9.34 | − | |
| Punicalin | C₃₄ H₂₂ O₂₂ | 782.0619 | 9.368 | + | + |
| Luteolin (isomer 2) | C₂₁ H₂₀ O₁₁ | 448.1011 | 9.779 | +/− | +/− |
| Chebulagic acid (isomer 2) | C₄₁ H₃₀ O₂₇ | 954.0978 | 9.847 | − | − |
| Casuarenin | C₄₁ H₂₈ O₂₆ | 936.0868 | 9.852 | − | − |
| Norstictic acid pentaacetate | C₂₈ H₂₄ O₁₅ | 600.1117 | 9.996 | +/− | + |
| 9,12,13-Trihydroxy-10,15- | C₁₈ H₃₂ O₅ | 328.2253 | 11.398 | − | |
| octadecadienoic acid | | | | | |
| Punicalin | C₃₄ H₂₂ O₂₂ | 782.0619 | 11.448 | + | |
| Jasmonic acid | C₁₂ H₁₈ O3 | 210.1257 | 11.536 | | + |
| Luteolin (isomer 3) | C₁₅ H₁₀ O₆ | 286.0484 | 11.864 | +/− | +/− |
| Quercetin | C₁₅ H₁₀ O₇ | 302.0434 | 11.91 | − | |
| 4,12-Dihydroxy-hexadecanoic acid | C₁₆ H₃₂ O₄ | 288.2306 | 11.913 | − | |
| Methyl-p-coumarate | C₁₀ H₁₀ O₃ | 178.0632 | 12.005 | − | |
| 9,12,13-Trihydroxy-10-octadecenoic | C₁₈ H₃₄ O₅ | 330.2412 | 12.491 | − | |
| acid | | | | | |
| Trimethyl ellagic acid (isomer 1) | C₁₇ H₁₂ O₈ | 344.0543 | 12.574 | +/− | +/− |
| methyl 9,12-dihydroxy- | C₁₉ H₃₄ O₅ | 342.2412 | 12.664 | − | |
| 13-oxo-10-octadecenoate | | | | | |
| Gingerol | C₁₇ H₂₆ O₄ | 294.1837 | 13.015 | − | − |
| Trimethyl ellagic acid (isomer 2) | C₁₇ H₁₂ O₈ | 344.0545 | 14.238 | +/− | +/− |
| Trimethyl ellagic acid (isomer 3) | C₁₇ H₁₂ O₈ | 344.0545 | 15.116 | +/− | +/− |
| 16-Hydroperoxy-9Z,12,14E- | C₁₈ H₃₀ O₄ | 310.2145 | 15.251 | − | |
| octadecatrienoic acid | | | | | |
| 9,13-Dihydroxy-11-octadecenoic acid | C₁₈ H₃₄ O₄ | 314.246 | 20.265 | − | − |
| Heptyl heptanoate | C₁₄ H₂₈ O₂ | 228.2091 | 20.996 | − | − |
| Palmitic acid | C₁₆ H₃₂ O₂ | 256.2413 | 23.869 | − | − |

In Table 3 above, + and − refers to the relevant ionisation mode in which the compound was detected. KLM=T. ferdinandiana leaf methanolic extract; KLE=T. ferdinandiana ethyl acetate extract.

The diversity of ellagitannins in the methanolic and ethyl acetate T. ferdianadiana leaf extracts was particularly noteworthy.

As well as from ellagic acid and the dehydrated and trimethylated derivatives, the more complex, higher molecular weight compounds (j) chebulinic acid and punicalin were also putatively identified and are likely to contribute to the C. perfringens growth inhibitory activity of these extracts.

Ellagitannins are considered to be identified as potent inhibitors of the growth of a broad panel of bacteria, with MIC values as low as 62.5 μg/ml.

The T. ferdinandiana extracts are shown to display low toxicity towards Artemia franciscana. Indeed, with the exception of the leaf ethyl acetate extract (MIC 767 μg/mL), the LC₅₀ values for all extracts were well in excess of 1000 μg/mL and are therefore nontoxic.

Bacillus Anthracis (B. Anthracis)

The ability to inhibit the growth of B. anthracis was investigated using a disc diffusion assay.

The minimum inhibitory concentration (MIC) values of the fruit and the leaf extracts were determined in order to quantify and compare their efficacies.

Toxicity was determined using an Artemia franciscana nauplii bioassay.

The most potent T. ferdinandiana fruit and leaf extracts were investigated using known non-targeted gas chromatography/mass spectrometry—GC-MS headspace analysis (with screening against a compound database) for the identification and characterisation of individual components in the crude T. ferdinandiana extracts.

Results: Solvent extractions of T. ferdinandiana fruit and leaf displayed good growth inhibitory activity in the disc diffusion assay against B. anthracis.

Fruit ethyl acetate and methanolic T. ferdinandiana leaf extracts were particularly potent growth inhibitors, with MIC values of 451 and 377 μg/mL respectively.

The fruit methanolic and chloroform extracts, as well as the aqueous leaf extracts, also were good inhibitors of B. anthracis growth (MIC values of 1800 and 1414 μg/mL respectively).

The aqueous fruit extract and leaf chloroform extracts had only low inhibitory activity.

All other extracts were completely devoid of growth inhibitory activity.

Furthermore, all of the extracts with growth inhibitory activity were nontoxic in the Artemia fransiscana bioassay, with LC50 values >1000 μg/mL. Non-biased GC-MS phytochemical analysis of the most active extracts (fruit ethyl acetate and methanolic leaf) putatively identified and highlighted several compounds that may contribute to the ability of these extracts to inhibit the growth of B. anthracis.

The low toxicity of the T. ferdinandiana fruit ethyl acetate and methanolic leaf extracts, as well as their potent growth inhibitory bioactivity against B. anthracis, indicates their previously unrealised suitability as medicinal agents in the treatment and prevention of anthrax.

Qualitative phytochemical studies: Phytochemical analysis of the extracts for the presence of alkaloids, anthraquinones, cardiac glycosides, flavonoids, phenolic compounds, phytosteroids, saponins, tannins and triterpenoids were conducted.

Antioxidant capacity: The antioxidant capacity of each sample was assessed using the DPPH free radical scavenging method with modifications.

Ascorbic acid (0-25 μg per well) was used as a reference and the absorbances were recorded at 515 nm.

All tests were completed alongside controls on each plate and all were performed in triplicate. The antioxidant capacity based on DPPH free radical scavenging ability was determined for each extract and expressed as μg ascorbic acid equivalents per gram of original plant material extracted.

Antibacterial screening: Environmental Bacillus anthracis screening: An environmental strain of Bacillus anthracis was isolated and identified. All growth studies were performed using a modified peptone/yeast extract (PYE) agar: 1 g/L peptone, 1.5 g/L yeast extract, 7.5 g/L NaCl, 1 g/L ammonium persulfate, 2.4 g/L HEPES buffer (pH 7.5) and 16g/L bacteriological agar when required. Incubation was at 30° C. and the stock culture was subcultured and maintained in PYE media at 4° C.

Evaluation of antimicrobial activity: Antimicrobial activity of all plant extracts was determined using a modified disc diffusion assay. Briefly, 100 μL of the test bacterium was grown in 10 mL of fresh nutrient broth media until they reached a count of ˜108 cells/mL.

A volume of 100 μL of the bacterial suspension was spread onto nutrient agar plates and extracts were tested for antibacterial activity using 5 mm sterilised filter paper discs. Discs were impregnated with 10 μL of the test sample, allowed to dry and placed onto the inoculated plates. The plates were allowed to stand at 4° C. for 2 hours before incubation at 30° C. for 24 hours.

The diameters of the inhibition zones were measured to the closest whole millimetre.

Each assay was performed in at least triplicate. Mean values (±SEM) are reported in this study. Standard discs of penicillin (2 μg) and ampicillin (10 μg) were obtained and used as positive controls for antibacterial activity. Filter discs impregnated with 10 μL of distilled water were used as a negative control.

Minimum inhibitory concentration (MIC) determination: The minimum inhibitory concentrations (MIC) of the extracts was determined as previously described.

Briefly, the plant extracts were diluted in deionised water and tested across a range of concentrations. Discs were impregnated with 10 μL of the extract dilutions, allowed to dry and placed onto inoculated plates.

The assay was performed as outlined above and graphs of the zone of inhibition versus concentration were plotted. MIC values were determined using linear regression.

Toxicity screening: Reference toxin for toxicity screening: Potassium dichromate (K₂Cr₂O₇) was prepared in distilled water (4 mg/mL) and serially diluted in artificial seawater for use in the Artemia franciscana nauplii bioassay.

Artemia franciscana nauplii toxicity screening: Toxicity was tested using a modified A. franciscana nauplii lethality assay. Briefly, 400 μL of seawater containing approximately 43 (mean 43.2, n=155, SD 14.5) A. franciscana nauplii were added to wells of a 48 well plate and immediately used in the bioassay.

A volume of 400 μL of reference toxin or the diluted plant extracts were transferred to the wells and incubated at 25±1° C. under artificial light (1000 Lux). A negative control (400 μL seawater) was run in triplicate for each plate. All treatments were performed in at least triplicate.

The wells were checked at regular intervals and the number of dead counted.

The nauplii were considered dead if no movement of the appendages was observed within 10 seconds. After 24 hours, all nauplii were sacrificed and counted to determine the total % mortality per well. The LC50 with 95% confidence limits for each treatment was calculated using probit analysis.

Non-targeted GC-MS head space analysis: Separation and quantification were performed using a mass selective detector system. Briefly, the system was equipped with an auto-sampler fitted with a solid phase micro-extraction fibre (SPME) handling system utilising a divinyl benzene/carbowax/polydimethylsiloxane (DVB/CAR/PDMS). Chromatographic separation was accomplished using a 5% phenyl, 95% dimethylpolysiloxane (30 m×0.25 mm id×0.25 um) capillary column. Helium (99.999%) was employed as a carrier gas at a flow rate of 0.79 ml/min. The injector temperature was set at 230° C.

Sampling utilised a SPME cycle which consisted of an agitation phase at 500 rpm for a period of 5 sec.

The fibre was exposed to the sample for 10 min to allow for absorption and then desorbed in the injection port for 1 min at 250° C. The initial column temperature was held at 30° C. for 2 min, increased to 140° C. for 5 min, then increased to 270° C. over a period of 3 mins and held at that temperature for the duration of the analysis.

The GC-MS interface was maintained at 200° C. with no signal acquired for a min after injection in split-less mode. The mass spectrometer was operated in the electron ionisation mode at 70 eV. The analytes were then recorded in total ion count (TIC) mode. The TIC was acquired after a min and for duration of 45 mins utilising a mass range of 45-450 m/z.

Statistical analysis: Data is expressed as the mean±SEM of at least three independent experiments.

Results

Liquid extraction yields and qualitative phytochemical screening: Extractions of the various dried T. ferdinandiana fruit and leaf plant materials (1 g) with various solvents yielded dried plant extracts ranging from 18 mg (hexane fruit extract) to 483 mg (aqueous fruit extract) (Table 4).

Methanolic and aqueous extracts gave significantly higher yields of dried extracted material compared to the chloroform, hexane and ethyl acetate counterparts, which gave low to moderate yields. The dried extracts were resuspended in 10 mL of deionised water (containing 1% DMSO), resulting in the extract concentrations shown in Table 4.

TABLE 4
Table 4: The mass of dried extracted material, the concentration after resuspension in deionised water,
qualitative phytochemical screenings and antioxidant capacities of the T ferdinandiana extracts:

| | | Concentration | Antioxidant Capacity | | | | | |
|---|---|---|---|---|---|---|---|---|
| | Mass of Dried | of Resuspended | (mg Ascorbic Acid | Total | Water Soluble | Water Insoluble | Cardiac | |
| Extract | Extract (mg) | Extract (mg/mL) | Equivalency) | Phenolics | Phenolics | Phenolics | Glycosides | Saponins |
| FW | 483 | 48.3 | 264 | +++ | +++ | +++ | − | + |
| FM | 359 | 35.9 | 660 | +++ | +++ | +++ | − | ++ |
| FC | 62 | 6.2 | 7 | + | − | − | − | − |
| FH | 18 | 1.8 | 1 | − | − | − | − | − |
| FE | 30 | 3 | 39 | ++ | ++ | + | − | + |
| LW | 471 | 47.1 | 340 | +++ | +++ | +++ | ++ | +++ |
| LM | 331 | 33.1 | 150 | +++ | +++ | +++ | +++ | ++ |
| LC | 59 | 5.9 | 5 | + | − | − | − | − |
| LH | 58 | 5.8 | 0.4 | + | − | − | − | − |
| LE | 59 | 5.9 | 22 | +++ | +++ | +++ | − | − |

| | | | Alkaloids | Alkaloids | | | Free | Combined |
|---|---|---|---|---|---|---|---|---|
| Extract | Triterpenes | Phytosteroids | (Mayer Test) | (Wagner Test) | Flavonoids | Tannins | Anthraquinones | Anthraquinones |
| FW | − | − | − | − | +++ | ++ | − | − |
| FM | + | − | + | + | +++ | ++ | − | − |
| FC | − | − | − | − | − | − | − | − |
| FH | − | − | − | − | − | − | − | − |
| FE | ++ | − | − | − | ++ | − | − | − |
| LW | ++ | − | − | − | ++ | +++ | + | + |
| LM | + | − | + | + | ++ | +++ | + | + |
| LC | − | − | − | − | − | − | − | − |
| LH | − | − | − | − | ++ | + | − | − |
| LE | − | − | − | − | ++ | ++ | − | − |

In Table 4 above, + + + indicates a large response; + + indicates a moderate response; + indicates a minor response; − indicates no response in the assay. FW=aqueous T. ferdinandiana fruit extract; FM=methanolic T. ferdinandiana fruit extract; FC=chloroform T. ferdinandiana fruit extract; FH=hexane T. ferdinandiana fruit extract; FE=ethyl acetate T. ferdinandiana fruit extract; LW=aqueous T. ferdinandiana leaf extract; LM=methanolic T. ferdinandiana leaf extract; LC=chloroform T. ferdinandiana leaf extract; LH=hexane T. ferdinandiana leaf extract; LE=ethyl acetate T. ferdinandiana leaf extract. Antioxidant capacity was determined by DPPH reduction and is expressed as mg ascorbic acid equivalence per g plant material extracted.

Antimicrobial activity: To determine the ability of the T. ferdinandiana fruit and leaf crude plant extracts to inhibit the growth of B. anthracis, aliquots (10 μL) of each extract were screened using a disc diffusion assay.

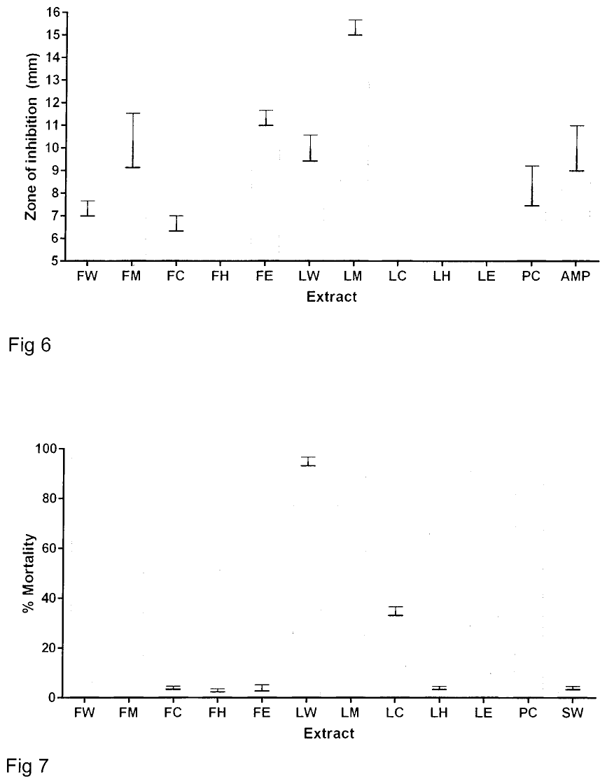

The bacterial growth was strongly inhibited by 7 of the 10 extracts screened (70%) (FIG. 6).

The methanolic leaf extract was the most potent inhibitor of B. anthracis growth (as judged by zone of inhibition), with inhibition zones of 15.3±0.6 mm. This compares favourably with the penicillin (2 μg) and ampicillin controls (10 μg), with zones of inhibition of 8.3±0.6 and 10.0±0.7 respectively.

The methanolic fruit extract as well as the ethyl acetate and aqueous leaf extracts also displayed good inhibition of B. anthracis growth, with 8 mm zones of inhibition.

In general, the leaf extracts were more potent inhibitors of B. anthracis growth than were their fruit extract counterparts.

The antimicrobial efficacy was further quantified through the determination of MIC values against the T. ferdinandiana extracts (Table 5).

Most of the extracts were effective at inhibiting B. anthracis growth, with MIC values <1000 μg/ml for several extracts (<10 μg impregnated in the disc).

The ethyl acetate fruit extract and the methanolic leaf extract were particularly potent, with MIC values of 451 μg/mL (approximately 4.5 μg infused into the disc) and 377 μg/mL (approximately 3.8 μg infused into the disc) respectively.

These results compare well with the growth inhibitory activity of the penicillin and ampicillin controls which were tested at 2 μg and 10 μg respectively.

The methanolic fruit extract was also a potent B. anthracis growth inhibitor (MIC value of 877 μg/ml).

Whilst less potent, the fruit chloroform and aqueous leaf extracts also had good growth inhibitory activity (MIC values of 1800 and 1414 μg/ml respectively).

In contrast, the aqueous fruit and hexane extracts, as well as the leaf chloroform hexane and ethyl acetate extracts, were not active, or were of only low efficacy in the assay.

Table 5 below shows minimum inhibitory concentration (μg/mL) of the T. ferdinandiana fruit and leaf extracts and LC50 values (μg/mL) in the Artemia nauplii bioassay.

TABLE 5

| Extract | MIC | LC50 |
|---|---|---|
| aqueous fruit extract | >10,000 | 2,080 |
| methanolic fruit extract | 877 | 2,115 |
| chloroform fruit extract | 1800 | — |
| hexane fruit extract | — | — |
| ethyl acetate fruit extract | 451 | — |
| aqueous leaf extract | 1414 | 1,330 |
| methanolic leaf extract | 377 | 1,133 |
| chloroform leaf extract | 5,000 | — |
| hexane leaf extract | — | 767 |
| ethyl acetate leaf extract | | |

Numbers indicate the mean MIC and LC50 values of triplicate determinations.
— indicates no inhibition.

Quantification of toxicity: All extracts were initially screened at 2000 μg/mL in the assay (see FIG. 7). For comparison, the reference toxin potassium dichromate (1000 μg/mL) was also tested in the bioassay.

The potassium dichromate reference toxin was rapid in its onset of mortality, inducing nauplii death within the first 3 hours of exposure and 100% mortality was evident following 4-5 hours (results not shown).

All methanolic and aqueous extracts showed >90% mortality rates at 24 hour, as did the ethyl acetate leaf extract. The remainder of the extracts showed <10% mortality rates at 24 hour, with the exception of the chloroform leaf extract.

To further quantify the effect of toxin concentration on the induction of mortality, the extracts were serially diluted in artificial seawater to test across a range of concentrations in the Artemia nauplii bioassay at 24 hours.

Table 5 shows the LC50 values of the T. ferdinandiana extracts towards A. franciscana. No LC50 values are reported for either of the chloroform and hexane extracts, nor for the ethyl acetate fruit extract, as less than 50% mortality was seen for all concentrations tested.

Extracts with an LC50 greater than 1000 μg/ml towards Artemia nauplii have been defined as being nontoxic in this assay.

As only the ethyl acetate fruit extract had a LC50 <1000 μg/ml, all other extracts were considered nontoxic. Whilst the LC50 value for leaf ethyl acetate extract is below 1000 pg/ml, the value of 767 μg/ml indicates low to moderate toxicity.

Non-targeted GC-MS headspace analysis of T. ferdinandiana fruit and leaf extracts: As the fruit ethyl acetate and methanolic leaf extracts had the greatest growth inhibitory efficacy against B. anthracis (as determined by MIC; see Table 5), they were deemed the most promising extracts for further phytochemical analysis. Optimised GC-MS parameters were developed and used to examine the phytochemical composition of these extracts.

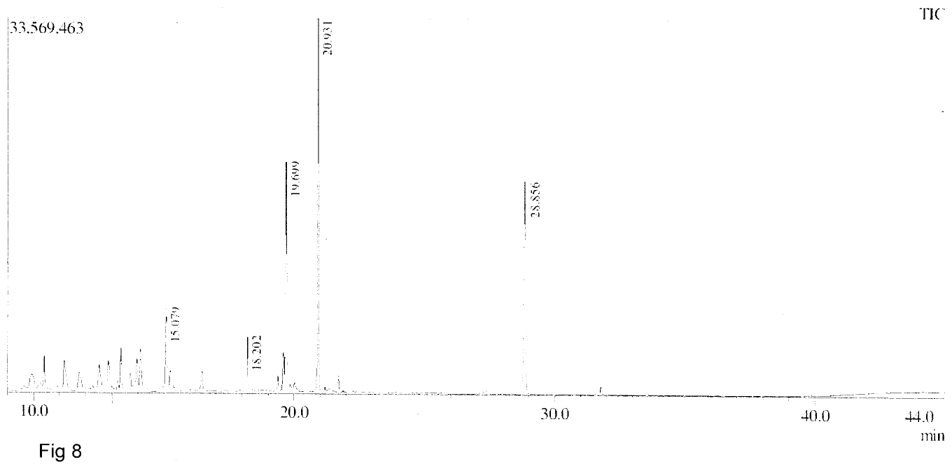

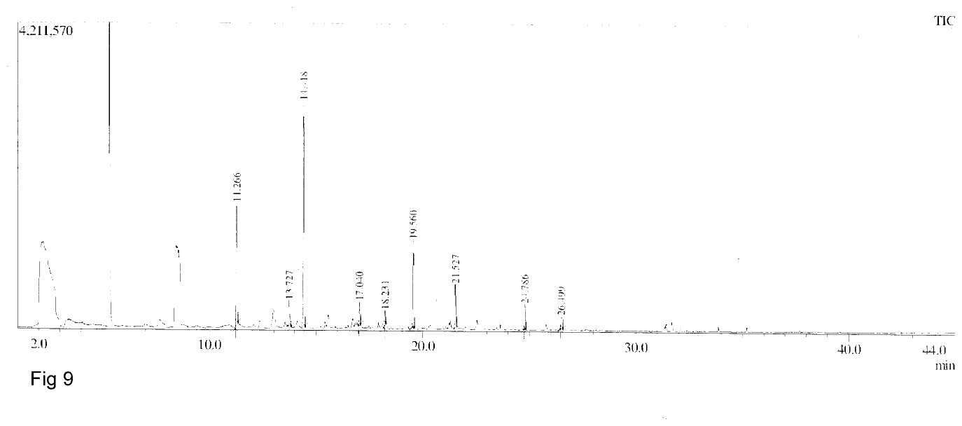

The resultant gas chromatograms for the fruit ethyl acetate and methanolic leaf extracts are presented in FIGS. 8 and 9 respectively. Several major peaks were noted in the fruit ethyl acetate extract at approximately 15.1 (3, 3-dimethyl-hexane, 7.1% relative abundance), 19.7 (2-methyl-2-phenyl-oxirane, 14.6% relative abundance), 20.9 (m-di-tert-butylbenzene, 22% relative abundance) and 28.9 min (3,5-bis(1,1-dimethylethyl)-phenol, 19.4% relative abundance). Numerous overlapping peaks were also evident in the middle stages of the chromatogram from 10-25 min. In total, 42 unique mass signals were noted for the T. ferdinandiana fruit ethyl acetate extract (Table 6). Putative empirical formulas and identifications were achieved for all of these compounds.

Table 6 below shows qualitative GC-MS analysis of the T. ferdinandiana fruit ethyl acetate extract, elucidation of empirical formulas and putative identification of each compound:

| | | | Retention | Relative |
|---|---|---|---|---|
| | Molecular | Empirical | Time | Abundance |
| Putative Identification | Mass | Formula | (min) | (% Area) |
| Ethanone, 1-(2-furanyl)- | 110 | C₆ H₆ O₂ | 10.202 | 0.9 |
| 2-Heptanone, 4-methyl- | 128 | C₈ H₁₆ O | 11.184 | 3.8 |
| Propanoic acid, 2- | 144 | C₅ H₈ N₂ O₃ | 11.8 | 1 |
| acetylhydrazono- | | | | |
| Tridecane | 184 | C₁₃ H₂₈ | 12.087 | 0.6 |
| Acetic acid, heptyl ester | 158 | C₉ H₁₈ O₂ | 12.263 | 0.2 |
| 3-Pentanol, 2,2-dimethyl- | 116 | C₇ H₁₆ O | 12.525 | 2.8 |
| 2-Heptanone, 4,6-dimethyl- | 142 | C₉ H₁₈ O | 12.866 | 3.2 |
| 3-Heptanol, 2-methyl- | 130 | C₈ H₁₈ O | 13.063 | 0.2 |
| 1-Octanol, 2-butyl- | 186 | C₁₂ H₂₆ O | 13.233 | 0.8 |
| Heptane, 3,3,5-trimethyl- | 142 | C₁₀ H₂₂ | 13.531 | 0.3 |
| Undecane | 156 | C₁₁ H₂₄ | 13.69 | 1.5 |
| Octane, 3,3-dimethyl- | 142 | C₁₀ H₂₂ | 13.96 | 2.5 |
| 1-Hexanol, 2-ethyl- | 130 | C₈ H₁₈ O | 14.105 | 3.8 |
| 1,3-Propanediamine, N,N- | 102 | C₅ H₁₄ N₂ | 14.3 | 0.3 |
| dimethyl- | | | | |
| Hexane, 3,3-dimethyl- | 114 | C₈ H₁₈ | 15.079 | 7.1 |
| 1 -Octanol | 130 | C₈ H₁₈ O | 15.422 | 0.3 |
| Propanoic acid, anhydride | 130 | C₆ H₁₀ O₃ | 15.625 | 0.2 |
| 1,3-Benzenediol, 4-ethyl- | 138 | C₈ H₁₀ O₂ | 16.034 | 0.4 |
| 1-Undecene, 4-methyl- | 168 | C₁₂ H₂₄ | 16.461 | 1.9 |
| Carbonic acid, nonyl prop-1- | 228 | C₁₃ H₂₄ O₃ | 17.241 | 0.2 |
| en-2-yl ester | | | | |
| Octanoic acid | 144 | C₈ H₁₆ O₂ | 18.481 | 0.8 |
| Oxalic acid, 6-ethyloct-3-yl | 314 | C₁₈ H₃₄ O₄ | 18.665 | 0.4 |
| isohexyl ester | | | | |
| 2-Octanol, 2,6-dimethyl- | 158 | C₁₀ H₂₂ O | 18.914 | 0.1 |
| Undecane, 4,7-dimethyl- | 184 | C₁₃ H₂₈ | 19.387 | 0.8 |
| Oxirane, 2-methyl-2-phenyl- | 134 | C₉ H₁₀ O | 19.699 | 14.6 |
| Nonane, 2,6-dimethyl- | 156 | C₁₁ H₂₄ | 19.803 | 0.6 |
| Dodecane, 4-methyl- | 184 | C₁₃ H₂₈ | 20.035 | 0.3 |
| Undecane, 2,4-dimethyl- | 184 | C₁₃ H₂₈ | 20.678 | 0.3 |
| 4-tert-Butylcyclohexyl methyl | 262 | C₁₃ H₂₇ O₃ P | 20.777 | 0.3 |
| ethylphosphonate | | | | |
| m-Di-tert-butylbenzene | 190 | C₁₄ H₂₂ | 20.931 | 22 |
| Hexane, 3,3-dimethyl- | 114 | C₈ H₁₈ | 21.08 | 0.2 |
| Nonadecane | 268 | C₁₉ H₄₀ | 21.703 | 1 |
| Butyl 2-butoxyacetate | 188 | C₁₀ H₂₀ O₃ | 21.841 | 0.7 |
| Octane, 2,3,6,7-tetramethyl- | 170 | C₁₂ H₂₆ | 22.087 | 0.1 |
| Hexane, 3,3-dimethyl- | 114 | C₈ H₁₈ | 22.959 | 0.2 |
| 1,1,6,6- | 180 | C₁₃ H₂₄ | 23.348 | 0.6 |
| Tetramethylspiro[4.4]nonane | | | | |
| 2,2,4-Trimethyl-1,3- | 286 | C₁₆ H₃₀ O₄ | 23.611 | 0.4 |
| pentanediol diisobuty | | | | |
| n-Decanoic acid | 172 | C₁₀ H₂₀ O₂ | 23.862 | 0.1 |
| Propanoic acid, 2-methyl-, | 216 | C₁₂ H₂₄ O₃ | 24.169 | 0.6 |
| 3-hydroxy-2,2 | | | | |
| 1-Dodecanol | 186 | C₁₂ H₂₆ O | 27.352 | 0.1 |
| Phenol, 3,5-bis(1,1- | 206 | C₁₄ H₂₂ O | 28.856 | 19.4 |
| dimethylethyl)- | | | | |
| 2,2,4-Trimethyl-1,3- | 286 | C₁₆ H₃₀ O₄ | 31.769 | 0.5 |
| pentanediol diisobutyrate | | | | |

The relative abundance expressed in this table 6 is a measure of the area under the peak expressed as a % of the total area under all chromatographic peaks

The gas chromatogram for the methanolic leaf extract (FIG. 9) had substantially fewer peaks evident than the fruit ethyl acetate extract (FIG. 8). In total, nineteen unique mass signals were noted in the methanolic leaf extract chromatogram.

Several major peaks were present at approximately 11.3 (methoxy-phenyl-oxime, 22.7% relative abundance), 13.7 (1-octen-3-ol, 2.4% relative abundance), 14.4 (2-(1,1-dimethylethoxy)-ethanol, 27.7% relative abundance), 19.5 (2-methyl-2-phenyl-oxirane, 11.4% relative abundance) and 21.5 min (3,5-dimethyl-benzaldehyde, 15.6% relative abundance).

Several small peaks were also evident throughout the chromatogram. Of the nineteen unique mass signals, putative empirical formulas and identifications were achieved for sixteen of these compounds.

Table 7 below shows a qualitative GC-MS analysis of the methanolic T. ferdinandiana leaf extract, elucidation of empirical formulas and putative identification of each compound.

TABLE 7

| | | | Retention | Relative |
|---|---|---|---|---|
| Putative | Molecular | Empirical | Time | Abundance |
| Identification | Mass | Formula | (mins) | (% Area) |
| Oxime-, methoxy- | 151 | C₈ H₉ NO₂ | 11.266 | 22.7 |
| phenyl-_{—} | | | | |
| 1-Octen-3-ol | 128 | C₈ H₁₆ O | 13.727 | 2.4 |
| Ethanol, 2-(1,1- | 118 | C₆ H₁₄ O₂ | 14.418 | 27.7 |
| dimethylethoxy)- | | | | |
| 1-Hexanol, 2-ethyl- | 130 | C₈ H₁₈ O | 15.373 | 1.1 |
| Cineole | 154 | C₁₀ H₁₈ O | 15.499 | 1.8 |
| Ethyl 2-(5-methyl-5- | 242 | C₁₃ H₂₂ O₄ | 16.879 | 1 |
| vinyltetrahydrofuran- | | | | |
| 2-yl carbonate | | | | |
| Nonanal | 142 | C₉ H₁₈ O | 17.04 | 1.3 |
| | | | 17.873 | 2.1 |
| | | | 18.231 | 0.6 |
| Oxirane, 2-methyl-2- | 134 | C₉ H₁₀ O | 19.56 | 11.4 |
| phenyl- | | | | |
| Ethyl benzoate | 150 | C₉ H₁₀ O₂ | 20.06 | 0.8 |
| 2-lsopropylidene-3- | | | | |
| methylhexa-3,5- | 150 | C₁₀ H₁₄ O | 21.031 | 0.5 |
| dienal | | | | |
| Decanal | 156 | C₁₀ H₂₀ O | 21.11 | 0.3 |
| Benzaldehyde, 3,5- | 134 | C₉ H₁₀ O | 21.527 | 15.6 |
| dimethyl- | | | 24.786 | 0.9 |
| Propanoic acid, 2- | 216 | C₁₂ H₂₄ O₃ | 26.499 | 2.2 |
| methyl-, 3-hydroxy- | | | | |
| 2,2,4 | | | | |
| 2,4-Di-tert- | 206 | C₁₄ H₂₂ O | 31.641 | 1 |
| butylphenol | | | | |
| Ethyl para- | 194 | C₁₁ H₁₄ O₃ | 32.054 | 5.9 |
| ethoxybenzoate | | | | |
| 2,2,4-Trimethyl-1,3- | 286 | C₁₆ H₃₀ O₄ | 33.822 | 0.7 |
| pentanediol | | | | |
| diisobutyrate | | | | |

The relative abundance expressed in table 7 is a measure of the area under the peak expressed as a % of the total area under all chromatographic peaks.

Qualitative GC-MS headspace analysis of the most potent B. anthracis growth inhibitory T. ferdinandiana extracts (fruit ethyl acetate and methanolic leaf extracts) identified a number of interesting compounds.

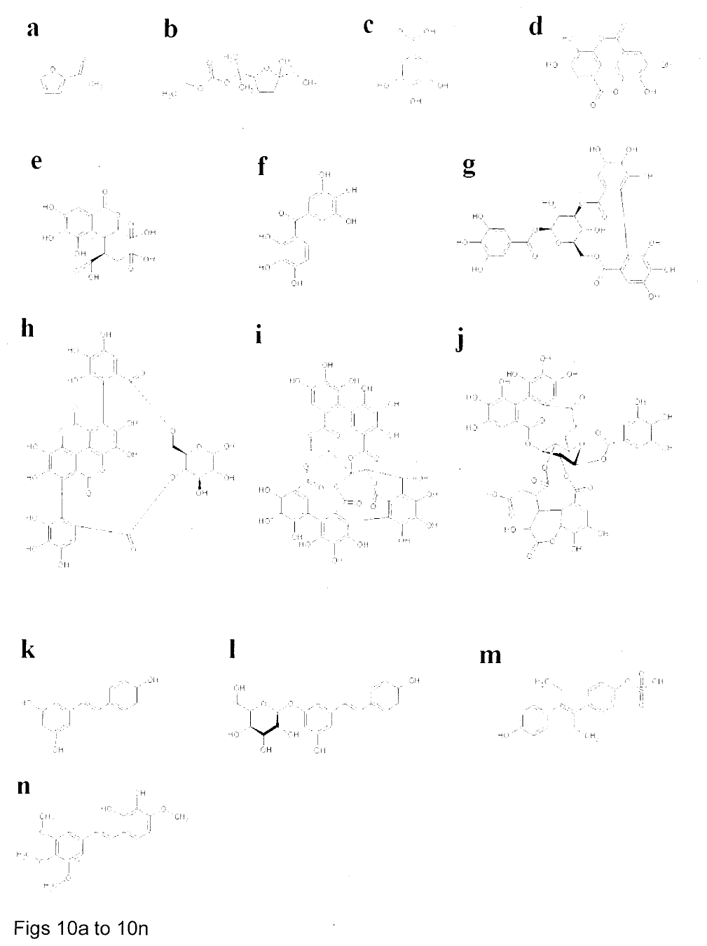

The presence of the furan compounds 1-(2-furanyl)-ethanone (FIG. 10a) and ethyl 2-(5-methyl-5-vinyltetrahydrofuran-2-yl) carbonate (FIG. 10b) are noteworthy. The nitro furans have particularly well studied antimicrobial mechanisms, acting via the inhibition of nucleic acid synthesis.

Similarly, synthetic furan derivatives (modified by the addition of a rhodanine moiety) are known to be potent inhibitors of the growth of a panel of multidrug resistant bacteria, with MIC values as low as 2 μg/mL against some species.

Reports of anti-bacterial activity for the two furan derivatives present in the T. ferdinandiana extracts are not known, and it is pertinent that the two furan derivatives are likely to contribute to the effectiveness of the present extracts.

It is likely that other phytochemical classes also contribute to the growth inhibitory properties of these extracts. Phytochemical screening indicates that polyphenolics, flavonoids, saponins, and terpenes were present in the T. ferdinandiana extracts.

Gallic (FIG. 5c) and ellagic acids (FIG. 5d) and their methylated derivatives, chebulic acid (FIG. 5e), galloyl pyrogallol (FIG. 5f), corilagen (FIG. 5g), punicalin (FIG. 5h), castalagin (FIG. 5i) and chebulagic acid (FIG. 5j) were detected in T. ferdinandiana extracts in each of those studies. These tannins have potent, broad spectrum growth inhibitory activity against a variety of bacterial species.

Gallotannins have particularly well reported inhibitory properties. They function via multiple mechanisms including interacting with both cell surface proteins and through interactions with intracellular enzymes.

Ellagitatannins also interact with cellular proteins and induce disruptions in bacterial cell walls.

Resveratrol (FIG. 10k) and the glycosylated resveratrol derivative piceid (FIG. 10l), diethylstilbestrol monosulfate (FIG. 10m) and combretastatin A1 (FIG. 10n) were putatively identified. Identification of combretastatin A1 was particularly interesting as combretastatins have attracted much recent interest due to their potent ability to block cancer cell progression and induce apoptosis by binding intracellular tubulin, thereby disrupting microtubule formation.

FIG. 10 (10a-n) show respective chemical structures of (a) 1-(2-furanyl)-ethanone, (b) ethyl 2-(5-methyl-5-vinyltetrahydrofuran-2-yl)carbonate, (c) gallic acid, (d) ellagic acid; (e) chebulic acid, (f) galloyl pyrogallol, (g) corilagen, (h) punicalin, (i) castalagin, (j) chebulagic acid, (k) resveratrol, (l) piceid, (m) diethylstilbestrol monosulfate, (n) combretastatin A1.

Several important terpenoids have also been identified in T. ferdinandiana extracts.

With the exception of the T. ferdinandiana ethyl acetate leaf extract, the findings reported here demonstrate that the T. ferdinandiana extracts were nontoxic towards Artemia franciscana nauplii, with LC50 values substantially >1000 μg/mL.

Extracts with LC50 values >1000 μg/mL towards Artemia nauplii are defined as being nontoxic. Even the ethyl acetate leaf extract which induced significant mortality was deemed low to moderate toxicity due to its moderate LC50 value.

Whilst toxicity investigations indicate that these extracts may be safe for use as B. anthracis growth inhibitors, studies using human cell lines are required to further evaluate the safety of these extracts.

T. ferdinandiana Extracts as Inhibitors of Giardia Proliferation and/or Control of Giardiasis.

By way of particular, though non-limiting, examples, inhibition of Giardia duodenalis proliferation by T. ferdinandiana extracts and pure compounds will hereinafter be described. It is to be understood that one or more forms of the present invention are not to be limited to control or inhibition of only Giardia duodenalis (aka Giardia lamblis and Giardia intestinalis) but of other Giardia and microbial/bacterial strains.

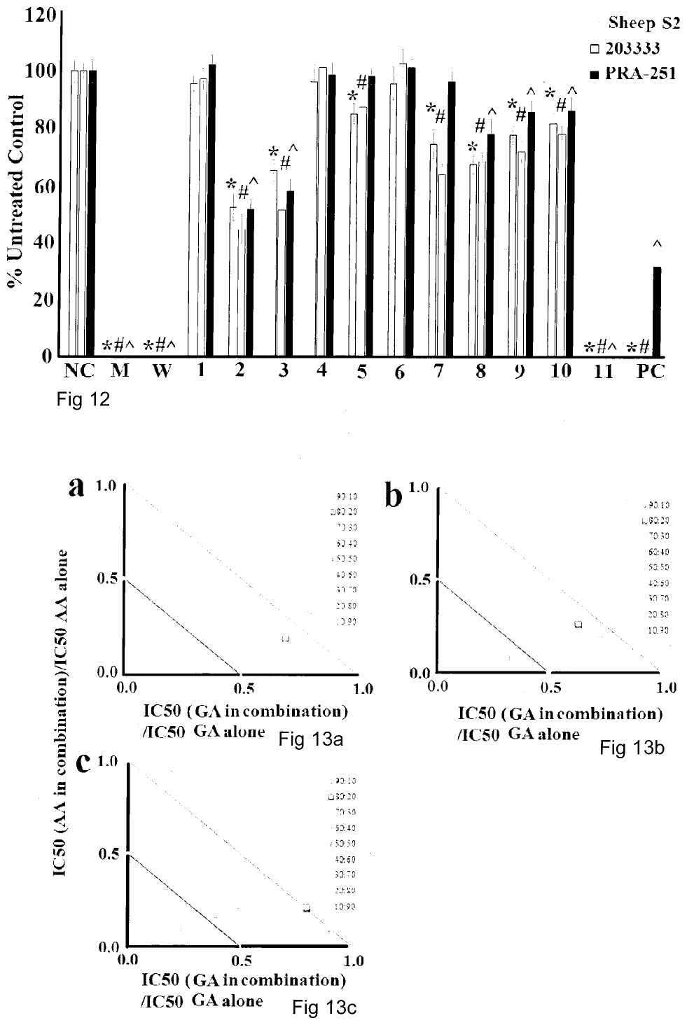

Inhibitory activity of T. ferdinandiana extracts and pure compounds have been tested, such as against three strains of Giardia duodenalis trophozoites measured as a percentage the untreated control, as shown by way of example in the test results shown in FIG. 12.

A panel of 11 compounds identified in T. ferdinandiana fruit extracts with potent G. duodenalis growth inhibitory activity have been investigated for the ability to inhibit G. duodenalis proliferation.

Eight of the 11 compounds inhibited the growth of all three G. duodenalis strains.

DPGA was the most potent antigiardial compound, with IC50 values as low as 126 μM (38 mg/mL). Notably, DPGA inhibited a metronidazole resistant G. duodenalis strain with similar potency as determined for the metronidazole sensitive strains.

Furthermore, the potency of DPGA was greatly potentiated when it was tested in combination with ascorbic acid, to approximately 17 μM (5 mg/mL) for the metronidazole sensitive G. duodenalis strains and 40 μM (12 mg/mL) for the resistant strain.

T. ferdinandiana tannins (gallic acid and chebulic acid) were also found to be inhibitors of G. duodenalis growth, with enhanced levels of activity when tested in combination with ascorbic acid.

All of the tested compounds (and their combinations with ascorbic acid) displayed low toxic and all compounds conformed to Lipinski's rules of 5 with few violations, indicating their potential as drug leads and chemotherapies for the treatment and prevention of giardiasis.

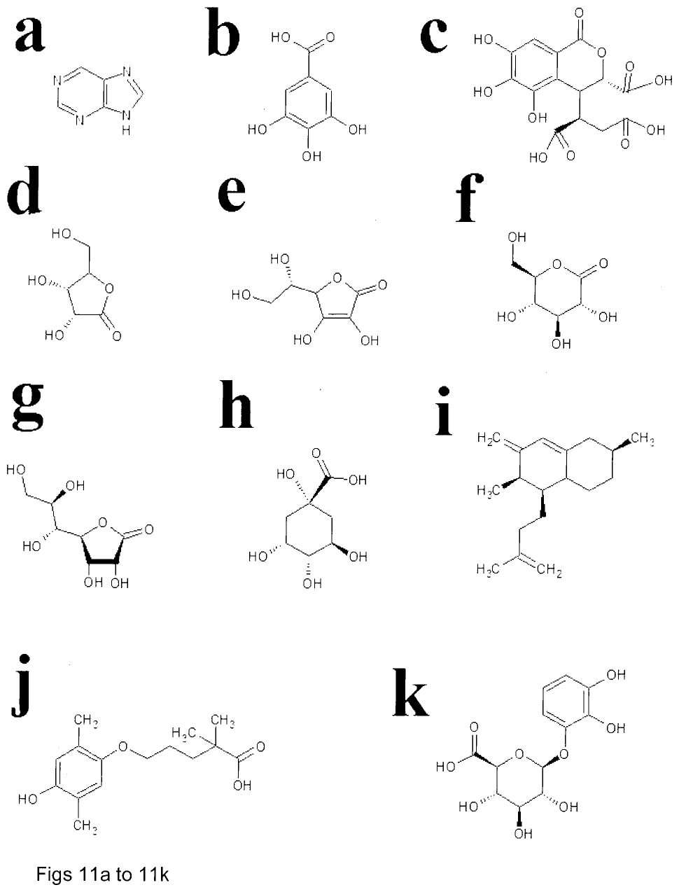

A purine analogue (FIG. 11a) was identified in all extracts with growth inhibitory activity.

Interestingly, numerous studies have reported that Giardia duodenalis are unable to synthesise their own purine or pyrimidine nucleotides and are reliant on salvage pathways to supply them with nucleotides for nucleic acid synthesis.

Furthermore, G. duodenalis are incapable of interconversion between purine nucleotides and therefore require the correct purine nucleotides for replication. Indeed, purine analogues inhibit the growth of G. duodenalis and have been highlighted as potential chemotherapeutic agents for giardiasis.

FIGS. 11a to 11k show respective chemical structures of the compounds reported in anti-proliferative methanolic and aqueous T. ferdinandiana fruit extracts: (a) purine; (b) gallic acid; (c) chebulic acid; (d) ribonolactone; (e) ascorbic acid; (f) gluconolactone; (g) glucohepatonic acid-1,4-lactone; (h) quinic acid, (i) eujavonic acid; (j) 5-(4-hydroxy-2,5-dimethylphenoxy)-2,2-dimethyl-pentanoic acid (HMDP); (k) 2,3-dihydroxyphenyl B-D-glucopyranosiduronic acid (DPGA).

An abundance of naturally occurring tannins in the bioactive T. ferdinandiana extracts is noted, with particularly high levels of gallic acid (FIG. 11b) and chebulic acid (FIG. 11c).

Gallotannins inhibit the growth of multiple microbial species via binding cell surface lipotoichoic acid and proline-rich membrane proteins, and by inhibiting glucosyltransferase enzymes.

Ribonolactone (FIG. 11d), ascorbic acid (FIG. 11e), gluconolactone (FIG. 11f) and glucohepatonic acid-1,4-lactone (FIG. 11g) present in extracts of T .ferdinandiana as lactone moieties is of value as many of the current anti-giardial chemotherapeutic drugs used are lactone containing compounds, particularly lactone substituted nitroimidazoles (e.g. metronidazole, secnidazole, tinidazole, ornidazole and albendazole).

Compounds containing a lactone moiety are understood to block the giardial lipid deacylation/reacylation pathways, thereby inhibiting proliferation. As Giardia spp. are unable to synthesise lipids by de novo pathways, they must use host gastrointestinal precursor lipids for the synthesis of membrane and cellular lipids by deacylation/reacylation reactions.

Thus, the lactone containing compounds in the T. ferdinandiana extracts can be understood to contribute to G. duodenalis growth inhibition via inhibition of lipid metabolism pathways.

Quinic acid (FIG. 11h) in the T. ferdinandiana extracts has been noted. Substituted quinic acid compounds can block leucyl-tRNA synthase activity in G. duodenalis cells. As aminoacyl-tRNA synthases are essential for translation of the genetic code by attaching the correct amino acid to each tRNA, blockage of leucyl-tRNA synthase activity results in ineffective Leu-tRNA production and thus the inhibition of protein synthesis. Therefore, quinic acid can be understood to also contribute to the antigiradial activity of the T. ferdianadiana fruit extracts.

Eujavonic acid (FIG. 11i), 5-(4-hydroxy-2,5-dimethylphenoxy)-2,2-dimethyl-pentanoic acid (HMDP) (FIGS. 11j) and 2,3-dihydroxyphenyl B-D-glucopyranosiduronic acid (DPGA) (FIG. 11k) are further antigiardial compounds.

Furthermore, as T. ferdinandiana fruit have a relatively high ascorbic acid content, ascorbic acid may be efficacious in altering and/or enhancing the growth inhibitory activity of the individual components.

Therefore, all compounds were also assessed in combination with ascorbic acid to quantify its effects on the activity of those components.

T. ferdinandiana fruit extraction yields and qualitative phytochemical screening.

Extraction of 1 g of dried T. ferdinandiana fruit with methanol and deionised water yielded relatively high masses of dried extracted material (370 μg/mL and 290 μg/mL for the methanolic and aqueous extracts respectively). The dried extracts were resuspended in 10 mL of deionised water (containing 0.5% DMSO) resulting in the extract concentrations shown in Table 8 below.

TABLE 8

| | | | | | | | | | | Toxicity | |
|---|---|---|---|---|---|---|---|---|---|---|---|
| | Mass of | | | | | | | | | | LC₅₀ in |
| | extracted | | | | | | | | Anti-giardial | LC₅₀ in | the HDF |
| | material | | | | | | | | IC₅₀ | ALA | assay |
| Extract | (mg) | Polyphenolics | Flavonoids | Phytosterols | Saponins | Triterpenoids | Tannins | Alkaloids | (ug/mL) | (ug/mL) | (ug/mL) |
| M | 370 | +++ | +++ | − | ++ | + | ++ | + | 740 | 1150 | 1450 |
| W | 290 | +++ | +++ | − | + | − | ++ | + | 143 | 1093 | 1540 |

+++ indicates a larqe response; ++ indicated a moderate response; + indicates a low response; − indicates no response. Values indicate the mean IC₅₀ or LC₅₀ values of three experiments each with triplicate determinations. M = methanolic extract; W = aqueous extract.

Qualitative phytochemical studies (Table 8) showed that both extracts contained high levels of phenolics and flavonoids, as well as moderate to high levels of tannins. Saponins were also present in low to moderate levels. Triterpenes and alkaloids were present in low levels.

Several of the pure T. ferdinandiana fruit compounds also significantly inhibited G. duodenalis trophozoite proliferation when tested at 300 μg/mL (FIG. 12).

DPGA was noted to be a particularly good growth inhibitor, blocking 100% of trophozoite growth.

DPGA was as effective against the metronidazole resistant G. duodenalis strain as it was against the sensitive strains, indicating that DPGA may block giardial growth by different mechanisms than metronidazole.

Several of the other compounds also significantly inhibited G. duodenalis trophozoite proliferation, albeit with lower efficacy. Gallic acid (˜50% inhibition of proliferation), chebulic acid (˜40% inhibition), quinic acid (˜30% inhibition), eujavonic acid (˜20% inhibition) and 5-(4-hydroxy-2,5-dimethylphenoxy)-2,2-dimethylpentanoic acid (˜20% inhibition) each inhibited all three G. duodenalis strain, including the metronidazole resistant strain.

Two of the other T. ferdinandiana fruit compounds (ascorbic acid, ˜15% inhibition; glucohelapatonic acid lactone, ˜30% inhibition) also significantly inhibited the metronidazole sensitive G. duodenalis strain, yet were ineffective inhibitors of the metronidazole resistant G. duodenalis strain.

Purine, ribolactone and gluconolactone did not significantly affect the growth of any of the G. duodenalis strains.

Methanol and water T. ferdinandiana fruit extracts displayed potent inhibitory activity, each inhibiting 100% of the Giardial growth (compared to the untreated control).

The efficacy of the extracts were further evaluated by determination of the concentration required to inhibit G. duodenalis growth by 50% (IC50). The water extract was a particularly good inhibitor of G. duodenalis proliferation, with an IC50 of 143 μg/mL. The methanol extract, whilst less potent, also displayed good anti-Giardial activity (704 μg/mL).

FIG. 12: Inhibitory activity of the T. ferdinandiana extracts and pure compounds against three strains of Giardia duodenalis trophozoites measured as a percentage the untreated control. NC=negative control; M=methanolic extract; W=water extract; 1=purine; 2=gallic acid; 3=chebulic acid; 4=ribolactone; 5=ascorbic acid; 6=gluconolactone; 7=glucohelapatonic acid lactone; 8=quinic acid; 9=eujavonic acid; 10=HMDP; 11=DPGA; PC=metronidazole control (50 μg/ml). Results are expressed as the mean±SEM of three independent experiments with internal triplicate determinations (n=9). *, # and ̂ indicate results that are significantly different to the untreated controls for the sheep S2, ATCC 203333 and ATCC PRA-251 G. duodenalis strains respectively (p<0.01).

Quantification of IC₅₀ for the Pure T. Ferdinndiana Compounds

The anti-proliferative activity of the pure T. ferdinandiana compounds was further tested over a range of concentrations to determine the IC₅₀ values against G. duodenalis trophozoites (Table 9).

TABLE 9

| | | | | | | | IC₅₀ (μg/mL) Values and | | | |
|---|---|---|---|---|---|---|---|---|---|---|
| | | | | | | | Class of Combination | | | |
| Compound alone or | Sheep S2 Strain | | | | | | ATCC203333 | | | |
| in combination | Single | | | | | Class of | Single | | | |
| with ascorbic acid | Compound | | Combination | | ΣFIC₅₀ | Interaction | Compound | | Combination | |
| Gallic acid | 1156 | (6795 μM) | 146 | (858 μM) | 0.15 | Synergy | 1368 | (8041 μM) | 228 | (1340 μM) |
| Chebulic acid | 1283 | (3602 μM) | 427 | (1199 μM) | 0.56 | Additive | 985 | (2765 μM) | 320 | (898 μM) |
| Glucohepatonic | 1746 | (9914 μM) | 1330 | (7552 μM) | 1.19 | independent | 2255 | (12804 μM) | 1585 | (9567 μM) |
| acid lactone | | | | | | | | | | |
| Quinic acid | 1172 | (6099 μM) | 418 | (2175 μM) | 0.51 | Additive | 1428 | (7431 μM) | 632 | (3289 μM) |
| Eujavonic acid | 1438 | (6038 μM) | 525 | (2204 μM) | 0.58 | Additive | 1683 | (7067 μM) | 695 | (2918 μM) |
| HMDP | 1835 | (6895 μM) | 725 | (2724 μM) | 0.69 | Additive | 1585 | (5955 μM) | 655 | (2461 μM) |
| DPGA | 47 | (156 μM) | 5 | (17 μM) | 0.11 | Synergy | 38 | (126 μM) | 6 | (20 μM) |

| | | | | | | | | |
|---|---|---|---|---|---|---|---|---|
| Ascorbic acid | 1869 | (10618 μM) | NA | NA | NA | 1906 | (10828 μM) | NA |

| | IC₅₀ (μg/mL) Values and Class of Combination | | | | | | | |
|---|---|---|---|---|---|---|---|---|
| Compound alone or | ATCC203333 | | ATCC PRA-251 | | | | | |
| in combination | | Class of | Single | | | | | Class of |
| with ascorbic acid | ΣFIC₅₀ | Interaction | Compound | | Combination | | ΣFIC₅₀ | Interaction |
| Gallic acid | 0.38 | Synergy | 1255 | (7377 μM) | 276 | (1522 μM) | 0.32 | Synergy |
| Chebulic acid | 0.6 | Additive | 1220 | (3425 μM) | 446 | (1252 μM) | 0.52 | Additive |
| Glucohepatonic | 1.57 | Independent | 3536 | (20089 μM) | 3207 | (18209 μM) | 1.25 | Independent |
| acid lactone | | | | | | | | |
| Quinic acid | 0.73 | Additive | 1755 | (9133 μM) | 882 | (4590 μM) | 0.95 | Additive |
| Eujavonic acid | 0.64 | Additive | 1850 | (7768 μM) | 878 | (3687 μM) | 0.72 | Additive |
| HMDP | 0.81 | Additive | 2032 | (7635 μM) | 1058 | (3975 μM) | 1.1 | Independent |
| DPGA | 0.18 | Synergy | 72 | (238 μM) | 12 | (40 μM) | 0.18 | Synergy |

| | | | | | | | |
|---|---|---|---|---|---|---|---|
| Ascorbic acid | NA | NA | 2850 | (16190 μM) | NA | NA | NA |

Restults are expressed as mean of three independent experiments with internal triplicate determinations (n = 9). Interactions classes are synergistic (ΣFIC₅₀ ≤ 0.5), additive (ΣFIC₅₀ > 0.5-1.0), independent (ΣFIC₅₀ > 1.0-4.0) or antagonistic (ΣFIC₅₀ > 4.0). NA = results not available.

Most of the compounds produced only moderate to low G. duodenalis inhibitory activity, with IC₅₀ values >1000 μg/mL. DPGA was a substantially more potent inhibitor of G. duodenalis proliferation than the other compounds, with IC₅₀ values for the different strains ranging from 38-72 μg/mL (126-238 μM). Interestingly, DPGA was a relatively minor component of the aqueous and methanolic T. ferdinandiana extracts, accounting for substantially less than 0.1% of the total extract's mass (results not shown) and it is therefore unlikely that DPGA alone would account for the strong activity of the crude aqueous extract (143 μg/mL). Instead, it is likely that other compounds in the extract synergise the activity of one or more of the anti-proliferative T. ferdinandiana compounds.

As T. ferdinandiana fruit has a very high ascorbic acid content, it is possible that ascorbic acid may have synergistic interactions with one or more of the the T. ferdinandiana compounds. Therefore, these compounds were further investigated in combination with ascorbic acid to identify any interactions which may occur.

Combinational Effects of the T. Ferdinndiana Compounds and Ascorbic Acid on G. Duodenalis Proliferation

A range of combinational effects were observed between T. ferdinandiana extract components and ascorbic acid (Table 9).

Of particular note, two combinations produced synergistic interactions (gallic acid+ascorbic acid; DPGA+ascorbic acid). Some combinations produced approximately 10 fold increases in activity compared to the activity of either compound alone. The increase in activity for DPGA in combination with ascorbic acid was particularly noteworthy against the sheep S2 G. duodenalis strain, with IC₅₀ values decreasing from 47 μg/mL (156 M) alone, to 5 μg/mL (17 μM) in combination with ascorbic acid. A similar increase in potency was recorded against the metronidazole sensitive reference G. duodenalis strain (ATCC203333), with a decrease of IC₅₀ from 38 μg/mL (126 μM) alone, to 6 μg/mL (20 μM) in combination with ascorbic acid. Whilst slightly less potent against the metronidazole resistant G. duodenalis strain (ATCC PRA-251), the DPGA+ascorbic acid combination also produced clinically relevant IC₅₀ values of 12 μg/mL (40 μM). Substantial increases in potency were also recorded for the gallic acid+ascorbic acid combinations against all G. duodenalis strains. The decrease in IC₅₀ against the sheep S2 strain from 1150 μg/mL (6795 μM) alone, to 146 μg/mL (858 μM) in combination with ascorbic acid was notable. This combination was also synergistic against the other G. duodenalis strains. Interestingly, the IC₅₀ values were similar between both the metronidazole sensitive and resistant G. duodenalis strains, both with IC₅₀ values of approximately 250 μg/mL (1469 μM).

The majority of the other combinations produced additive effects. These combinations may therefore also be beneficial in the treatment of giardiasis, as they produce enhanced efficacy over either component when used separately.

A further combination (glucohepatonic acid lactone) was non-interactive. Whilst this combination does not provide any significant therapeutic benefit above that of either compound alone, the components also do not antagonise each other's effects and therefore it would not be detrimental if the two components were to be administered concurrently. Notably, none of the combinations produced antagonistic effects.

Synergistic Interactions Between Gallic Acid and Ascorbic Acid

As the gallic acid/ascorbic acid combination induced a synergistic interaction (Table 9), the combination was further examined using isobologram analysis across a range of gallic acid:ascorbic acid ratios to identify the ideal ratios to obtain synergy.

Similar susceptibility profiles were evident against all three G. duodenalis strains.

In all cases, the data correlated more closely with the gallic acid axis than with the ascorbic acid axis, indicating that the anti-proliferative activity is most reliant on the gallic acid.

However, whilst ratios containing between 30-60% gallic acid induced synergistic responses, the lower (20%) and higher ratios (70%) generally produced additive effects.

As these responses are greater than either of the individual components alone, they would therefore be beneficial for the treatment of giardiasis.

As synergy was determined using the ΣFIC₅₀ formula, synergy is defined as a response at least 4 times greater than that of the individual components alone. Thus the ratios which induce synergistic responses are far preferable as antigiardial therapies compared to the other ratios. Therefore, the ideal gallic acid/ascorbic acid ratios for the treatment of giardiasis can preferably include the combinations containing 30-60% gallic acid.

As shown by way of FIGS. 13a to 13c, Isobolograms for combinations of gallic acid and ascorbic acid tested at various ratios against (a) the sheep S2, (b) reference metronidazole sensitive (ATCC203333) and (c) reference metronidazole resistant (ATCC PRA-251) G. duodenalis strains. GA=gallic acid; AA=ascorbic acid. Results represent mean FIC₅₀ values of three independent experiments, each consisting of 3 replicates (i.e. 9 data points for each ratio). Ratios lying on or underneath the 0.5/0.5 line are considered to be synergistic (Σ FIC₅₀≤0.5). Any points between the 0.5/0.5 and 1.0/1.0 lines are deemed to be additive (Σ FIC₅₀>0.5-1.0).

Synergistic Interactions Between 2,3-Dihydroxyphenyl-B-Gloucopyranosiduronic Acid (DPGA) and Ascorbic Acid

DPGA also induced synergistic G. duodenalis growth inhibition when tested in combination with ascorbic acid (Table 9).

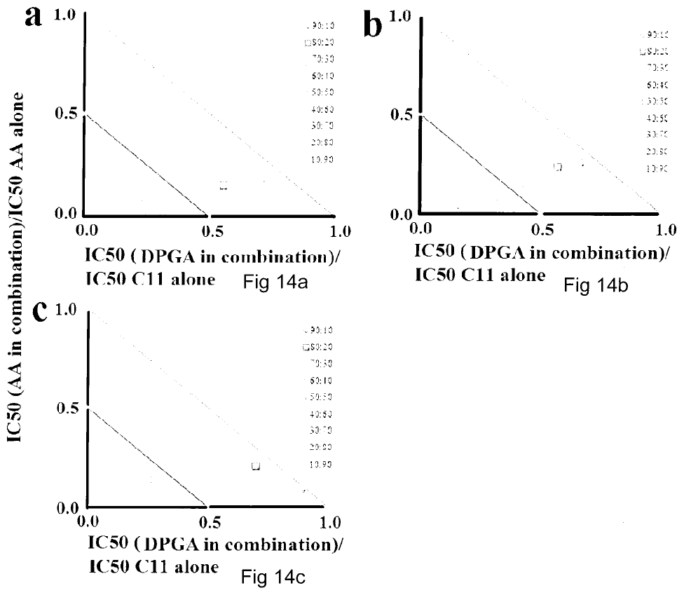

The association between the growth inhibitory activity and the DPGA axis was even more pronounced than for gallic acid (FIGS. 14a to 14c), indicating that this compound is more important than ascorbic acid for the anti-proliferative activity of this combination. This is consistent with the IC₅₀ data for the compounds which reports the IC₅₀ of DPGA as approximately 5% of the IC₅₀ of ascorbic acid (Table 9). Thus, DPGA is approximately a 20 times more potent G. duodenalis growth inhibitor than ascorbic acid when the components were tested separately. Interestingly, all combinations containing ≤60% DPGA produced synergistic inhibition of the growth for the metronidazole sensitive G. duodenalis strains (FIGS. 14a and 14b). Therefore, ≥40% ascorbic acid is required to effectively synergise the effects of DPGA.

The growth inhibition isobologram against the metronidaxole resistant G. duodenalis strain displays a different trend (FIG. 14c). The majority of the combination ratios produced additive interactions against this strain. These ratios would still be beneficial for treating giardiasis as the growth inhibitory activity of the combination is greater than that of either component alone. However, whilst the treatment efficacy is increased for these ratios, the increase in relatively minor. In contrast, combinations containing 30-50% DPGA had substantially increased efficacy (≥4 fold increases in potency compared to the sum of the compounds tested alone). Therefore, the ideal synergistic ratio for the treatment and prevention of giardiasis against the metronidazole resistant G. duodenalis strain was identified to be 30-50% DPGA in combination with ascorbic acid.

FIGS. 14a to 14c show isobolograms for combinations of DPGA and ascorbic acid tested at various ratios against (a) the sheep S2, (b) reference metronidazole sensitive (ATCC203333) and (c) reference metronidazole resistant (ATCC PRA-251) G. duodenalis strains. DPGA =2,3-dihydroxyphenyl-B-gloucopyranosiduronic acid; AA=ascorbic acid. Results represent mean FIC₅₀ values of three independent experiments, each consisting of 3 replicates (i.e. 9 data points for each ratio). Ratios lying on or underneath the 0.5/0.5 line are considered to be synergistic (Σ FIC₅₀≤0.5). Any points between the 0.5/0.5 and 1.0/1.0 lines are deemed to be additive (Σ FIC₅₀>0.5-1.0).

Quantification of Toxicity

All extracts were screened across a range of concentrations using both the Artemia nauplii lethality assay (ALA) and a human dermal fibroblast assay (HDF) (Table 10).

For comparison, the reference toxin potassium dichromate (1000 μg/mL) was also tested. No LC₅₀ values are reported for purine, ribolactone, gluconolactone, glucohepatonic acid lactone, quinic acid, eujavonic acid, HMDP, or DPGA as less than 50% mortality was seen for all concentrations of these compounds tested in both assays.

All of these compounds were therefore deemed to be nontoxic. In contrast, gallic acid, chebulic acid and ascorbic acid displayed apparent toxicity in both assays following 24 hours exposure. However, it is noteworthy that the toxicity detected in our study generally correlated with acidic components. Acidic pH can suppress the rate of mitochondrial protein synthesis and potentially be fatal to the growth and development of both Artemia nauplii and HDF cells. Indeed, previous studies have reported that extracts high in ascorbic acid can provide fallacious toxicity determinations. Thus, this assay may have overestimated the toxicity of these compounds.

TABLE 10
Table 10: Toxicity of the T. ferdinandiana compounds alone and in
combination with ascorbic acid determined by Artemia lethality
assay (ALA) and human dermal fibroblast (HDF) cytotoxicity assay.

| Toxicity (μg/mL) | |
|---|---|
| | Compound/ascorbic |
| Compound Alone | acid combination |

| Compound | ALA | HDF | ALA | HDF |
|---|---|---|---|---|
| Purine | CND | CND | NT | NT |
| Gallic acid | 132 | 320 | 147 | 336 |
| Chebulic acid | 165 | 380 | 224 | 375 |
| Ribolactone | CND | CND | NT | NT |
| Ascorbic acid | 203 | 358 | NA | NT |
| Gluconolactone | CND | CND | NT | NT |
| Glucohepatonic | CND | CND | >500 | >500 |
| acid lactone | | | | |
| Quinic acid | CND | CND | >500 | >500 |
| Eujavonic acid | CND | CND | >500 | >500 |
| HMDP | CND | CND | >500 | >500 |
| DPGA | CND | CND | >500 | >500 |
| PC | 37 | 42 | NT | NT |

PC = potassium dichromate; CND = could not determine as the mortality or % inhibition did not exceed 50% at any concentration tested; NT = not tested.

Therapeutic Index and Drug Like Properties

To determine the suitability of the T. ferdinandiana compounds as therapeutic agents, their drug-like properties were examined with reference to Lipinsky's rules of five.

All of the compounds had ≤10 H bond acceptors, molecular weights <500 Da and octanol-water coefficients ≤5. The majority of the compounds also had ≤5 H bond donors.

The only compounds that violated this rule (chebulic acid and DPGA) included the compound with the greatest G. duodenalis anti-proliferative activity (DPGA), both alone and in combination with ascorbic acid. Both DPGA and chebulic acid have 6 H bond donors and therefore exceed Lipinsky's rules of five by one H bond donor. However, given their conformity in all other categories, these compounds were deemed to have good drug-like properties.

The therapeutic index (TI) was also calculated for the pure compounds and combinations. We were unable to calculate TI's for purine, ribolactone, gluconolactone, glucohepatonic acid lactone, quinic acid, eujavonic acid, HMDP and DPGA as none of these compounds displayed toxicity at any concentration tested. However, with the exception of DPGA, these compounds generally displayed only low G. duodenalis anti-proliferative activity and were therefore of little use therapeutically. For DPGA, this lack of apparent toxicity indicates that the compound would have a high TI and therefore be a promising drug-lead. If the dose range that DPGA was tested over was extended to test higher concentrations to determine an LC₅₀, the TI would be relatively high.

An interesting trend was noted for the TI of gallic acid. The TI of this compound alone was relatively low (0.3) due to its apparent toxicity, indicating that it may have limited therapeutic potential. However, when the TI of gallic acid was determined in combination with ascorbic acid, it had increased substantially to 2.3. Thus, it is likely that ascorbic acid may provide dual benefits in combination with DPGA: it may synergise the anti-proliferative activity of DPGA, as well as protecting the cells against its toxicity.

TABLE 11
Drug like properties and therapeutic index of the T. ferdinandiana
compounds alone and in combination with ascorbic acid.

| | ≤5 H bond | ≤10 H bond | MW ≤500 | Octanol-water | Therapeutic Index | Therapeutic Index in |
|---|---|---|---|---|---|---|
| Compound | donors | acceptors | Da | coefficient ≤5 | of Compound | combination with AA |
| Purine | Yes | Yes | Yes | Yes | TBT | NT |
| Gallic acid | Yes | Yes | Yes | Yes | 0.3 | 2.3 |
| Chebulic acid | No | Yes | Yes | Yes | 0.3 | 0.9 |
| Ribolactone | Yes | Yes | Yes | Yes | TBT | NT |
| Ascorbic acid | Yes | Yes | Yes | Yes | 0.2 | NA |
| Gluconolactone | Yes | Yes | Yes | Yes | TBT | NT |
| Glucohepatonic | Yes | Yes | Yes | Yes | TBT | TBT |
| acid lactone | | | | | | |
| quinic acid | Yes | Yes | Yes | Yes | TBT | TBT |
| eujavonic acid | Yes | Yes | Yes | Yes | TBT | TBT |
| HMDP | Yes | Yes | Yes | Yes | TBT | TBT |
| DPGA | No | Yes | Yes | Yes | TBT | TBT |

TBT = the therapeutic index could not be determined as the toxicity was too low to determine an LC₅₀; NT = not tested in combination as the TI of the compound alone was inactive.

1. A composition for use in treating bacterial infection in humans or animals, the composition including a medicament containing an extract derived from Terminalia ferdinandiana (T. ferdinandiana) as an antimicrobial or antibacterial agent.

2. The composition of claim 1, including T. ferdinandiana leaf extract.

3. The composition of claim 2, further including an extract of T. ferdinandiana fruit in addition to the extract of T. ferdinandiana leaf.

4. The composition of claim 2, wherein the T. ferdinandiana leaf extract includes one or more of a methanolic or ethanolic extract, aqueous extract, ethyl acetate extract, chloroform extract or hexane extract.

5. The composition of claim 2, wherein the T. ferdinandiana leaf extract includes a proportion of at least one antioxidant.

6. The composition of claim 5, wherein the at least one antioxidant includes one or more of an ellagic acid or trimethyl ellagic acid.

7. The composition according to claim 1, wherein the composition, the medicament or the extract is provided in pill form, capsule form, or as a liquid.

8. The composition according to claim 1, including at least one tannin and/or at least one flavone and/or including one of or a combination of two or more of, chebulic acid, corilagen, chebulinic acid and chebulagic acid.

9. (canceled)

10. The composition according to claim 1, including at least one flavone or flavonoid.

11. The composition according to claim 1, for use in treating B. anthracis or C. perfringens or Giardia infection in humans or animals.

12. A medicament including an extract of Terminalia ferdinandiana (T. ferdinandiana) as an antimicrobial or antibacterial agent for use in treating bacterial infection in humans or animals.

13. The medicament of claim 12, provided for use in treating B. anthracis or C. perfringens or Giardia infection in humans or animals.

14. An extract of Terminalia ferdinandiana (T. ferdinandiana) for use in a medicament for treatment of microbial or bacterial infection in humans or animals.

15. The extract of claim 14, for use in treating B. anthracis or C. perfringens or Giardia infection in humans or animals.

16-17. (canceled)

18. The composition of claim 3, wherein the T. ferdinandiana leaf extract includes one or more of a methanolic or ethanolic extract, aqueous extract, ethyl acetate extract, chloroform extract or hexane extract.

19. The composition of claim 2, wherein the composition, the medicament or the extract is provided in pill form, capsule form, or as a liquid.

20. The composition according to claim 2, for use in treating B. anthracis or C. perfringens or Giardia infection in humans or animals.

21. The composition of claim 3, for use in treating B. anthracis or C. perfringens or Giardia infection in humans or animals.

22. The medicament of claim 12, including an extract of T. ferdinandiana leaf or including an extract of T. ferdinandiana leaf and an extract of T. ferdinandiana fruit.

23. The extract of claim 14, including an extract of T. ferdinandiana leaf, or including an extract of T. ferdinandiana leaf and an extract of T. ferdinandiana fruit.
